(19) United States
(12) Patent Application Publication
(10) Pub. No.: US 2019/0175557 A1
(43) Pub. Date: Jun. 13, 2019

(54) COMPOUNDS USEFUL FOR DECREASING INTERFERON LEVEL

(71) Applicants: CENTRE NATIONAL de la RECHERCHE SCIENTIFIQUE, Paris (FR); UNIVERSITE PARIS DESCARTES, Paris (FR)

(72) Inventors: Nicolas Pietrancosta, Paris (FR); Nikaïa Smith, Nice (FR); Jean-Philippe Herbeuval, Paris (FR)

(21) Appl. No.: 16/310,146

(22) PCT Filed: Jun. 16, 2017
(86) PCT No.: PCT/EP2017/064820

§ 371 Date: Dec. 14, 2018

(30) Foreign Application Priority Data
Jun. 16, 2016 (FR) 16305735.9

(51) Int. Cl. A61K 31/417; C07K 16/28; A61K 31/4045; A61K 31/429; A61K 31/4174; A61P 31/18; A61P 31/16; A61P 19/02; A61P 29/00; G01N 33/50; G01N 33/566; G16C 20/64; G16B 35/20

(52) U.S. Cl. CPC A61K 31/417; C07K 2317/21; A61K 31/4045; A61K 31/429; A61K 31/4174; A61P 31/18; A61P 31/16; A61P 19/02; A61P 29/00; G01N 33/5041; G01N 33/566; G16C 20/64; G16B 35/20; C07K 2317/24; C07K 2317/565; C07K 16/2866

(57) ABSTRACT

The invention relates to a CXCR4 receptor-binding compound for use for decreasing interferon (IFN) level in an individual.

### FIELD OF THE INVENTION

The present invention relates to CXCR4 receptor-binding compounds for use for decreasing interferon (IFN) level in an individual.

### BACKGROUND OF THE INVENTION

Interferons (IFN) mediate immune defence against viral infections. However, an overproduction of IFN, either inherited or acquired, leading to high IFN level, may be the cause of various disorders, such as autoimmune diseases or interferonopathies, which notably include Aicardi-Goutières syndrome, familial chilblain lupus, spondyenchondromatosis, Proteasome-associated auto-inflammatory syndrome (PRASS) and Singleton-Merten syndrome.

Current treatment of interferonopathies are mostly symptomatic and based on glucocorticoids (Munoz et al. (2015) Annales de dermatologie et de vénérologie 142:653-663). Besides, in complement to corticoids, physiotherapy sessions and psychological care are an integral part of the prevention of these disorders.

However, corticoids-based treatments have several side effects such as weight gain, hormonal disturbances, high blood pressure, growth-retardation in children, digestive disorders, sleeping disorders or mood disorders.

More generally, treatments currently available for interferonopathies are principally aimed at alleviating the symptoms rather than treating the underlying causes of the disease and are unable to maintain a long-lasting remission.

Accordingly, there is a need for an alternative to these therapies, which would be effective to effectively cure interferonopathies in addition to treating the symptoms.

### SUMMARY OF THE INVENTION

The present invention arises from the unexpected finding, by the inventors, that amines inhibit interferon (IFN) production by virus-stimulated plasmacytoid dendritic cells (pDC) in vitro and in vivo in an Influenza A-infected mouse model. Similarly, the inventors have shown that this inhibitory effect could be extended to monocytes, Natural Killer (NK) cells as well as to other cytokines, such as TNF-α, or interleukins such as IL-6, IL-8 or IL-10. The inventors have further identified the C—X—C chemokine receptor 4 (CXCR4) as the unexpected receptor used by amines to inhibit pDC production of IFN as well as the previously unknown binding site mediating this effect.

Thus, the present invention relates to a CXCR4 receptor-binding compound for use for decreasing a cytokine level, in particular interferon (IFN) level, in an individual, provided the CXCR4 receptor-binding compound is different from histamine.

In an embodiment, the invention relates to the CXCR4 receptor-binding compound for use according to the invention, for inhibiting cytokine secretion, in particular IFN secretion, by immune cells, in particular plasmacytoid dendritic cells, monocytes and Natural Killer (NK) cells.

In another embodiment, the invention relates to the CXCR4 receptor-binding compound for use according to the invention in the prevention or treatment of interferonopathies.

The present invention also relates to a method for decreasing, cytokine level, in particular interferon (IFN) level, in an individual, comprising administering to the individual an effective amount of at least one CXCR4 receptor-binding compound, provided the CXCR4 receptor-binding compound is different from histamine.

The present invention also relates to a method for inhibiting cytokine secretion, in particular IFN secretion, by immune cells, in particular plasmacytoid dendritic cells, monocytes and NK cells, in an individual, comprising administering to the individual an effective amount of at least one CXCR4 receptor-binding compound, provided the CXCR4 receptor-binding compound is different from histamine.

The present invention further relates to a method for the prevention or treatment of interferonopathies, comprising administering to the individual a prophylactically or therapeutically effective amount of at least one CXCR4 receptor-binding compound according to the invention, provided the CXCR4 receptor-binding compound is different from histamine.

The invention also relates to the in vitro use of a CXCR4 receptor-binding compound according to the invention, for inhibiting cytokine secretion, in particular IFN secretion, by immune cells, in particular plasmacytoid dendritic cells, monocytes and NK cells, provided the CXCR4 receptor-binding compound is different from histamine.

The present invention also relates to an in vitro method for inhibiting cytokine secretion, in particular IFN secretion, by immunes cells, in particular plasmacytoid dendritic cells, monocytes and NK cells, comprising contacting immune cells, in particular plasmacytoid dendritic cells, monocytes and NK cells with a CXCR4 receptor-binding compound according to the invention, provided the CXCR4 receptor-binding compound is different from histamine.

The invention also relates to an in vitro screening method for identifying compounds for decreasing cytokine level, in particular IFN level, in an individual from candidate compounds, wherein the candidate compounds are CXCR4 receptor-binding compounds as defined above.

The invention also relates to an in vitro screening method for identifying compounds for decreasing IFN level in an individual from candidate compounds, comprising the steps of:

contacting blood cells with a candidate compound;

determining the level of secretion of IFN by the contacted blood cells;

comparing the determined level of secretion of IFN to the level of expression of IFN by blood cells contacted by a reference compound;

selecting the candidate compound which have a decreased, increased or similar level of expression of IFN with respect to the reference compound, thereby identifying a compound for decreasing IFN level, wherein the reference compound is a CXCR4 receptor-binding compound according to the invention, in particular the 12G5 antibody or a compound of formula (II) as defined below, more particularly FFN102 or FFN511.

The invention also relates to an in vitro screening method for identifying compounds for decreasing cytokine level, in particular IFN level, in an individual from candidate compounds, comprising:

binding a CXRC4 receptor with a detectable CXCR4 receptor-binding compound as defined above;

contacting the CXCR4 receptor bound to the detectable CXCR4 receptor-binding compound with a candidate compound;

selecting the candidate compound which decreases the binding of the detectable CXCR4 receptor-binding compound to the CXCR4 receptor, thereby identifying a compound for decreasing IFN level.

The invention also relates to an in silico method for screening compounds useful for decreasing cytokine level, in particular IFN level, in an individual from candidate compounds, or for designing compounds useful for decreasing cytokine level, in particular IFN level, in an individual, comprising a computer-implemented step of determining if a designed compound or a candidate compound interacts with at least 8 amino acids of a CXCR4 receptor represented by SEQ ID NO: 1, wherein the amino acids are selected from the group consisting of tryptophan 94, tryptophan 102, aspartic acid 97, aspartic acid 187, tyrosine 116, tyrosine 190, arginine 183, isoleucine 185, valine 112, cysteine 186 and glutamic acid 288.

### DETAILED DESCRIPTION OF THE INVENTION

As intended herein, the term “comprising” has the meaning of “including” or “containing”, which means that when an object “comprises” one or several elements, other elements than those mentioned may also be included in the object. In contrast, when an object is said to “consist of” one or several elements, the object is limited to the listed elements and cannot include other elements than those mentioned.

### CXCR4 Receptor-Binding Compound

As is known in the art, the “CXCR4 receptor” is the C—X—C chemokine receptor type 4 also known as fusin or CD184. As intended herein, the expression “CXCR4 receptor” is equivalent to “CXCR4”. Preferably, the CXCR4 receptor according to the invention is a human CXCR4 receptor. CXCR4 is notably represented by SEQ ID NO: 1.

A CXCR4 receptor-binding compound according to the invention can either be known in the art to bind to CXCR4 or it can be determined that it binds to CXCR4. Determining that a compound binds to CXCR4 can be performed by numerous ways known to one of skill in the art. By way of example, CXCR4 binding is assessed by flow cytometry analysis of cells expressing CXCR4 contacted with a compound to be assessed using an anti-CXCR4 antibody, such as the 12G5 antibody. This procedure is explained in more details in the following Example.

Preferably, the CXCR4 receptor-binding compound according to the invention comprises from 1 to 45 carbon atoms and at least one amine group positively charged at a pH from 6 to 8, in particular at a pH from 7.0 to 7.8, more particularly at a physiological blood pH of a human individual.

Preferably also, the CXCR4 receptor-binding compound according to the invention interacts with at least 5, 6, 7, 8, 9, 10 or 11 amino acids of a CXCR4 receptor represented by SEQ ID NO: 1, wherein the amino acids are selected from the group consisting of tryptophan 94, tryptophan 102, aspartic acid 97, aspartic acid 187, tyrosine 116, tyrosine 190, arginine 183, isoleucine 185, valine 112, cysteine 186 and glutamic acid 288.

The above-defined amino acids have been identified by the present inventors as defining the binding site on the CXCR4 receptor responsible for decreasing IFN secretion by immune cells, in particular plasmacytoid dendritic cells, monocytes and NK cells. Besides, as should be clear to one of skill in the art, SEQ ID NO: 1 is only meant as a reference sequence to unequivocally define the positions of the amino acids of the CXCR4 receptor involved in the binding the CXCR4 receptor-binding compound according to the invention. Accordingly, SEQ ID NO: 1 is not meant to limit the CXCR4 receptors according to the invention. Indeed, the CXCR4 receptor-binding compounds according to the invention can also bind to the above-defined amino acids in variants, mutants or truncated forms of the CXCR4 receptor or in proteins or polypeptides comprising the CXCR4 receptor, which may change the absolute position of the amino acids in said variants, mutants or truncated forms or proteins or polypeptides, but not their function.

The CXCR4 receptor-binding compound according to the invention may in particular be a natural amine or a synthetic amine, a monoamine or a polyamine.

In an embodiment of the invention, the CXCR4 receptor-binding compound according to the invention is a natural amine and is preferably selected from the group consisting of serotonin, dopamine, L-dopa, spermine and spermidine. These natural amines are well known to one of skilled in the art and are represented by the following structures:

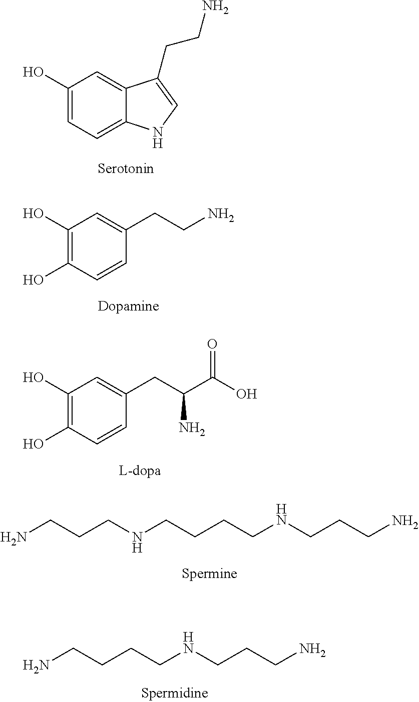 [SMILES: NCCc1c[nH]c2ccc(O)cc12 ; NCCc1ccc(O)c(O)c1 ; N[C@@H](Cc1ccc(O)c(O)c1)C(=O)O ; NCCCNCCCCNCCCN ; NCCCCNCCCN]

Histamine is represented by the following formula:

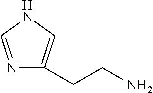

In another embodiment of the invention, the CXCR4 receptor-binding compound according to the invention is selected from the group consisting of an anti-CXCR4 receptor antibody, antibody fragment, scFv antibody, or aptamer.

As should be clear to one of skill in the art, the anti-CXCR4 receptor antibody, antibody fragment, scFv antibody, or aptamer according to the invention are all specifically directed against the CXCR4 receptor, more particularly against a site of the CXCR4 receptor defined by at least 5, 6, 7, 8, 9, 10 or 11 amino acids of a CXCR4 receptor represented by SEQ ID NO: 1, wherein the amino acids are selected from the group consisting of tryptophan 94, tryptophan 102, aspartic acid 97, aspartic acid 187, tyrosine 116, tyrosine 190, arginine 183, isoleucine 185, valine 112, cysteine 186 and glutamic acid 288.

As intended herein, a compound is said to be “specifically directed against” a target when the compound binds to the target without substantially binding to an unrelated target, e.g. for a protein, a non-homologous target.

As understood herein, an “antibody” according to the invention may be a monoclonal or a polyclonal antibody. Preferably, the antibody according to the invention is a monoclonal antibody (mAb) and the antibody fragments are monoclonal antibody fragments. Preferably also, the antibody according to the invention is a humanized antibody and the antibody fragments according to the invention are fragments of a humanized antibody.

Methods for producing antibodies, in particular monoclonal antibodies, directed against a specific target are well known to one of skilled in the art.

Preferably, an anti-CXCR4 receptor antibody according to the invention is the monoclonal anti-CXCR4 receptor antibody 12G5. This anti-CXCR4 receptor antibody is well known in the art, is notably described in Endres et al. (1996) Cell 87:745-756 and is commercially available. Preferably also, an anti-CXCR4 receptor antibody according to the invention is a humanized 12G5 antibody or a human antibody onto which have been grafted at least one complex determining region (CDR), and more preferably all the CDRs, of the 12G5 antibody.

The antibody fragment according to the invention can be of any type known to one of skilled in the art retaining the antigen-binding part of the antibody. In particular, the antibody fragment according to the invention selected from the group consisting of the Fab fragment, the Fab′ fragment or the F(ab′)2 fragment. Such fragments, and ways of obtaining them, are well known to one of skilled in the art. Preferably, the antibody fragment according to the invention is a 12G5 antibody fragment.

A single-chain variable fragment (scFv) antibody comprises the respective variable regions of the heavy (V_{H}) and the light (V_{L}) chains of an antibody, which are joined together by a peptide linker. The scFv antibody according to the invention can be obtained by numerous methods well known to one of skilled in the art.

Aptamers are single-stranded oligonucleotides molecules, DNA or RNA, preferably RNA. The aptamers according to the invention can be notably be obtained by the well-known systematic evolution of ligands by exponential enrichment (SELEX) method.

In an embodiment, the CXCR4 receptor-binding compound according to the invention is a compound of the following formula (I):

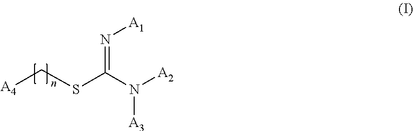

wherein:
n is an integer from 1 to 6,
A₁, A₂ and A₃, which may be identical or different, represent:
   a hydrogen atom, or
   an alkyl group having from 1 to 12 carbon atoms, optionally substituted by at least one hydroxyl group, a halogen atom, a carbonitril group, a trifluoromethyl group, an amine group, an urea, or an O-alkyl or S-alkyl group having from 1 to 12 carbon atoms, or
   an heterocycle, heteroaryl, aryl, arylalkyl or alkylaryl group having from 3 to 12 carbon atoms, optionally substituted by at least one hydroxyl group, a halogen atom, a carbonitril group, a trifluoromethyl group, an amine group, an urea, or an O-alkyl or S-alkyl having from 1 to 12 carbon atoms; and
A₄ represents an aryl, heteroaryl, arylalkyl or alkylaryl group having from 3 to 20 carbon atoms optionally substituted by at least one hydroxyl group, a halogen atom, a carbonitril group, a trifluoromethyl group, an amine group, an urea group, or an O-alkyl or S-alkyl group having from 1 to 12 carbon atoms;
   or a pharmaceutically acceptable salt and/or hydrate thereof.

Preferably, the compound of formula (I) as defined above is selected from the group consisting of clobenpropit (CB) and IT1t:

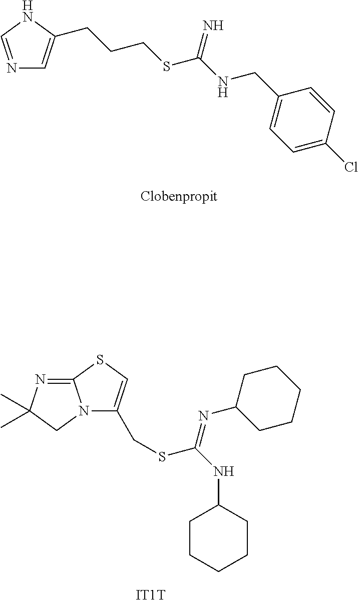 [SMILES: N=C(NCc1ccc(Cl)cc1)SCCCc1cnc[nH]1 ; CC1(C)CN2C(CS/C(=N/C3CCCCC3)NC3CCCCC3)=CSC2=N1 ; "IT1T"]

Preferably also, the CXCR4 receptor-binding compound according to the invention is a compound of formula (I) as defined above with the exception of clobenpropit.

In an embodiment of the invention, the CXCR4 receptor-binding compound according to the invention is a compound of formula (I) as defined above wherein:
A₁, A₂ and A₃, which may be identical or different, represent:
   a hydrogen atom, or
   an alkyl group having from 1 to 12 carbon atoms, optionally substituted by at least one hydroxyl group or a halogen atom, or
   an aryl, arylalkyl or alkylaryl group having from 3 to 12 carbon atoms, optionally substituted by at least one hydroxyl group, a halogen atom, or an O-alkyl or S-alkyl having from 1 to 12 carbon atoms; and
A₄ represents an aryl or heteroaryl group having from 3 to 12 carbon atoms optionally substituted by at least one hydroxyl group, a halogen atom, or an O-alkyl or S-alkyl group having from 1 to 12 carbon atoms, provided that A₄ is different from imidazole.

Compounds of formula (I) according to the invention can be readily synthesized by one of skill in the art, as is in particular described in Thoma et al. (2008) J. Med. Chem. 51: 7915-7920 and Van der Goot et al. European Journal of Medicinal Chemistry, 27: 511-157.

In another embodiment, the CXCR4 receptor-binding compound according to the invention is a compound of the following formula (II):

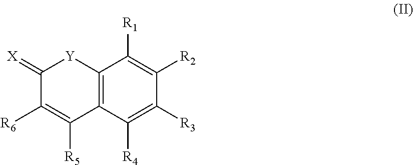

wherein
R₁, R₂, R₃, and R₄, which may be identical or different, represent a hydrogen atom, a halogen atom, a hydroxyl group, an alkyl group having from 1 to 12 carbon atoms, optionally substituted by at least one hydroxyl group, an amine group or a halogen atom, wherein R₁ and R₂, and/or R₂ and R₃ and/or R₃ and R₄ can be included in a same cycle;
X and Y, which may be identical or different, represent S or 0;
R₅ and R₆, which may be identical or different, represent a hydrogen atom or an alkyl group having from 1 to 5 carbon atoms substituted by at least one amine group, provided at least one of R₅ and R₆ represents an alkyl group having from 1 to 5 carbon atoms substituted by at least one amine group;
   or a pharmaceutically acceptable salt and/or hydrate thereof.

Preferably, the compound of formula (II) defined above is selected from the group consisting of FFN102 and FFN511.

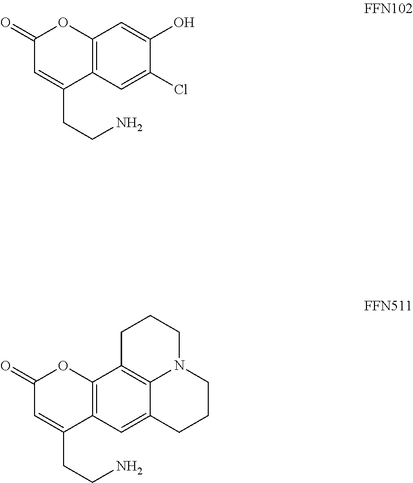 [SMILES: NCCc1cc(=O)oc2cc(O)c(Cl)cc12 ; NCCc1cc(=O)oc2c3c4c(cc12)CCCN4CCC3]

Advantageously, compounds of formula (II), in particular FFN102 and FFN5111, are fluorescent. Accordingly, such compounds can be used to assess binding to the CXCR4 receptor, for instance in competition studies.

Compounds of formula (II) according to the invention can be readily synthesized by one of skill in the art, as is in particular described in Gubernator et al (2009) Science, 324: 1441-1444 and Lee et al (2010) Journal of the American Chemical Society, 132: 8828-8830.

In yet another embodiment, the CXCR4 receptor-biding compound according to the invention is a compound of the following formula (III):

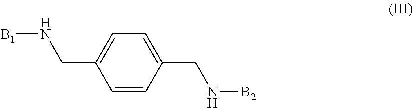

wherein
B₁ and B₂, which are identical or different, represent:
   an aryl or heteroaryl group having from 3 to 6 carbon atoms, optionally substituted by a hydroxyl group, a halogen atom, an alkoxy group, a thioalkoxy group, a CF₃ group, a CN group, a —NR₇R₈ group, an amide or an alkyl, S-alkyl or O-alkyl group having from 1 to 6 carbon atoms, or
   a cycloalkyl or heterocycloalkyl group having from 3 to 6 carbon atoms, optionally substituted by, a hydroxyl group, a halogen atom, an alkoxy group, a thioalkoxy group, a CF₃ group, a CN group, a —NR₇R₈ group or an alkyl, S-alkyl or O-alkyl group having from 1 to 6 carbon atoms,
   wherein R₇ and R₈ which are identical or different, represent H, an alkyl group having from 1 to 6 carbon atoms or a heterocycloalkyl group having from 3 to 6 carbon atoms;
      or a pharmaceutically acceptable salt thereof and/or hydrate thereof.

Preferably, the compound of formula (III) as defined above is selected from the compounds shown in FIG. 19 of the article of Debnath et al. (2013) Theranostics 3:47-75.

Compounds of formula (III) according to the invention can be readily synthesized by one of skill in the art, as is in particular described in Debnath et al. (2013) Theranostics 3:47-75 pages 66-67.

In still another embodiment, the CXCR4 receptor-biding compound according to the invention is a compound of the following formula (IV):

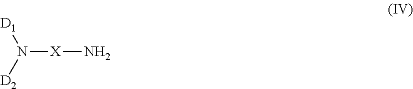

wherein
D₁ and D₂, which may be identical or different, represent:
   an alkyl group having from 1 to 6 carbon atoms, optionally substituted by at least one hydroxyl group, a halogen atom, a CF₃ group, a CN group, an amine group, or an alkyl, O-alkyl or S-alkyl group having from 1 to 12 carbon atoms, or
   an aryl, heteroaryl, cycloalkyl, aheterocycloalkyl, an alkylaryl, alkylheteroaryl or an alkylheteropolyaryl group having from 3 to 12 carbon atoms, optionally substituted by at least one hydroxyl group, a halogen atom, a CF₃ group, a CN group, an amine group, or an alkyl, O-alkyl or S-alkyl group having from 1 to 12 carbon atoms; or
   D₁ and D₂ are linked together to form a N-containing aryl or heteroaryl group having from 3 to 12 carbon atoms and optionally substituted by at least one amine group optionally substituted by an alkylheteroaryl group having from 3 to 12 carbon atoms, and
X represents:
   an alkyl group having from 1 to 6 carbon atoms, or
   R₉—Y—R₁₀— wherein, R₉ and R₁₀ which are identical or different represent an alkyl group having from 1 to 6 carbon atoms and Y represents an aryl or heteroaryl group having from 3 to 6 carbon atoms, optionally substituted by a halogen atom, a hydroxyl group, an amide group, an amine group, an alkoxy group, an ester group, a CF₃ group, a CN group or an alkyl, O-alkyl or S-alkyl group having from 1 to 6 carbon atoms optionally substituted by a hydroxyl group, an amine group or an O-alkyl group having from 1 to 6 carbon atoms;
      or a pharmaceutically acceptable salt thereof and/or hydrate thereof.

Preferably, the CXCR4 receptor-binding compound according to the invention is a compound of formula (IV) as defined above wherein:
D₁ and D₂, which may be identical or different, represent an aryl or heteroaryl group having from 3 to 12 carbon atoms, optionally substituted by a hydroxyl group or an alkyl group having from 1 to 6 carbon atoms,
X represents an alkyl group having from 1 to 6 carbon atoms,
   or a pharmaceutically acceptable salt and/or hydrate thereof.

or a pharmaceutically acceptable salt thereof and/or hydrate thereof.

Preferably, the compound of formula (IV) as defined above is selected form the compounds shown in FIG. 9 and FIG. 16 of the article of Debnath et al. (2013) Theranostics 3:47-75.

Preferably also, the compound of formula (IV) as defined above is represented by the following formula (V):

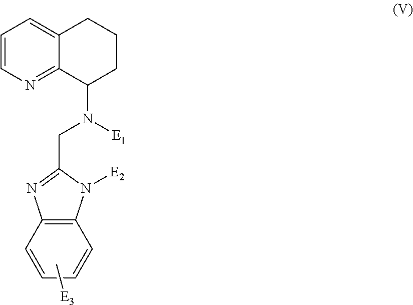

wherein:
E₁ represents an alkyl group having from 1 to 12 carbon atoms, or a heteroaryl group having from 3 to 12 carbon atoms, and
E₂ represents a heteroalkyl group having from 1 to 12 carbon atoms, substituted by an amine group, and
E₃ represents a heteroalkyl group having from 1 to 12 carbon atoms;
   or a pharmaceutically acceptable salt and/or hydrate thereof.

Most preferably, the compound of formula (IV) as defined above is selected form the group consisting of compounds represented by the following structures:

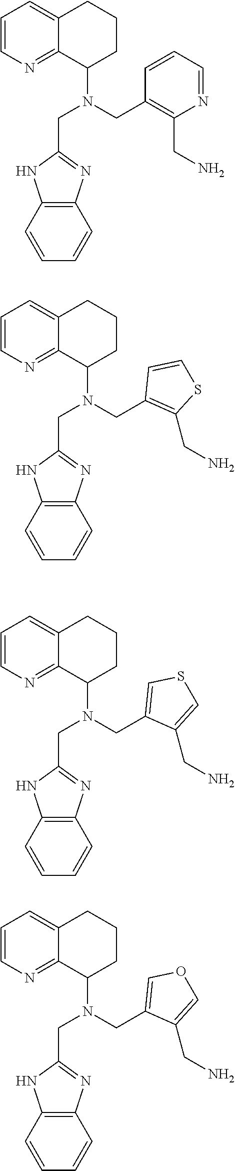

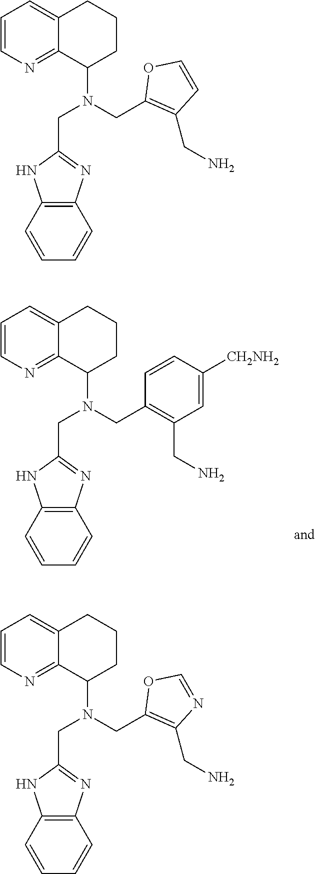

Preferably, the compound of formula (IV) according to the invention is AMD070:

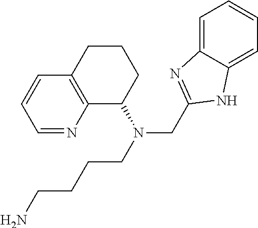

Compounds of formula (IV) according to the invention can be readily synthesized by one of skill in the art, as is in particular described in Miller et al. (2010) Bioorg. Med. Chem. Lett., 20: 3026-30.

The pharmaceutically acceptable salt and/or hydrate of compounds of formula (I), (II), (Ill), and (IV) will appear obviously to one of skilled in the art. Preferably, the pharmaceutically acceptable salt and/or hydrate of compounds of formula (I), (II), (Ill), and (IV) are selected from the group consisting of hydrobromide, hydrochloride, dihydrobromide and dihydrochloride.

As intended herein, the term “alkyl” refers to linear, branched or cyclic alkyl groups.

As intended herein, the term “aryl” denotes an aromatic group comprising at least one aromatic ring.

As intended herein, the term “heteroaryl” denotes an aryl comprising at least one heteroatom preferably selected from the group consisting of O, P, N, S and Si, which is more preferably N.

As intended herein, the term “heteroalkyl”, in particular “heterocycloalkyl”, denotes an alkyl, in particular a cycloalkyl, comprising at least one heteroatom preferably selected from the group consisting of O, P, N, S and Si, which is more preferably N.

As intended herein the term “alkylaryl” denotes an alkyl group substituted by at least one aryl group.

As intended herein the term “arylalkyl” denotes an aryl group substituted by at least one alkyl group.

The halogen atom according to the invention can be of any type known to one of skilled in the art. Preferably, the halogen atom according to the invention is selected from the group consisting of F, Cl, Br and I.

Preferably, the CXCR4 receptor-binding compound according to the invention is selected from the group consisting of IT1t, clobenpropit, FFN102, FFN511 and AMD070. More preferably, the CXCR4 receptor-binding compound according to the invention is selected from the group consisting of IT1t, FFN102, FFN511 and AMD070.

### Prevention and Treatment

The cytokine according to the invention can be a pro-inflammatory or an anti-inflammatory cytokine.

Preferably, the cytokine according to the invention is TNF-α, an interleukin, such as IL-6, IL-8 or IL-10, or an interferon, more preferably selected from the group consisting of a type I interferon, also denoted IFN-I, a type II interferon, also denoted IFN-II, and a type III interferon, also denoted IFN-III. More preferably, the IFN according to the invention is selected from the group consisting of IFN-α, IFN-β, IFN-ω, IFN-γ and IFN-λ. Most preferably, the interferon according to the invention is IFN-α.

As will be clear to one of skill in the art the level of cytokine or interferon, preferably IFN-I, IFN-II or IFN-III, to be decreased according to the invention is preferably an abnormal or pathological level, which is more preferably abnormally or pathologically elevated.

As intended herein an abnormally or pathologically elevated level of interferon, in particular IFN-I, IFN-II or IFN-III, is preferably a level of interferon, i.e. a concentration of interferon, above 1 u.i/ml, in particular in a human individual.

Preferably, inhibition of cytokine secretion, in particular IFN secretion, according to the invention, relates to inhibition of secretion by immune cells, i.e. cells of the immune system, more preferably inhibition of the secretion by dendritic cells, in particular plasmacytoid dendritic cells, cells of monocyte/macrophage lineage, in particular monocytes, and Natural Killer (NK) cells.

Preferably, the prevention or treatment according to the invention relates to the prevention or treatment of at least one symptom, disorder or disease associated with or caused by an over-production or an excess of IFN, in particular IFN-I, IFN-II or IFN-III, or a high or elevated IFN level, in particular IFN-I, IFN-II or IFN-III level. The over-production or excess of IFN, in particular IFN-I, IFN-II or IFN-III, or high or elevated IFN level, in particular IFN-I, IFN-II or IFN-III level, can be either acquired, for instance as a consequence of a viral infection, or inherited, for instance as a genetic disorder.

More preferably, the present invention relates to the prevention or treatment of an interferonopathy, in particular a type-I interferonopathy, i.e. an interferonopathy associated to IFN-I.

Type-I interferonopathies are generally defined as a group of Mendelian disorders characterised by a physiopathology: the up-regulation of type I interferons. Interferonopathies are notably described in Munoz et al. (2015) Annales de Dermatologie et de vènèrèologie, 142: 653-663.

Interferonopathies according to the invention are preferably selected from the group consisting of Aicardi-Goutiéres syndrome, familial chilblain lupus, spondyenchondromatosis, Systemic lupus erythematosus, in particular associated to a deleterious heterozygous mutation of the TREX gene, Sting-associated vasculopathy, Proteasome-associated auto-inflammatory syndrome (PRAAS) and Singleton-Merten syndrome.

More preferably also, the present invention relates to the prevention or treatment of diseases caused by, or associated to, an over-production, an up-regulation, an excess, or a high, elevated or above-normal level, of IFN-II, in particular autoimmune diseases such as those described in Baccala et al., (2005) Immunological Reviews, 204: 9-26.

Preferably, diseases caused by, or associated to, an over-production, an up-regulation, an excess, or a high, elevated or above-normal level, of IFN-II, are selected from the group consisting of Systemic lupus erythematosus, rheumatoid arthritis and type I diabetes mellitus.

Preferably also, the present invention relates to the prevention or treatment of an autoimmune disease, in particular selected from Systemic lupus erythematosus, Sjogren's syndrome, Aicardi-Goutières, myositis, in particular polymyositis and dermatomyositis, psoriasis, systemic sclerosis, type I diabetes mellitus, autoimmune thyroid disease, rheumatoid arthritis, Crohn's disease and multiple sclerosis, as well as atherosclerosis. More preferably, the present invention relates to the prevention or treatment of rheumatoid arthritis or systemic lupus erythematosus, or psoriasis. All these latter diseases are known to be associated to an IFN, or an IFN is known to be one of their aetiological agent as is indicated in for example in Niewold (2014) Frontiers in Immunology 5:1-2, Goosens et al. (2010) Cell Metabolism 12:142-153, Greenberg (2010) Arthritis Research & Therapy 12(Suppl 1): S4, and Pollard et al. (2013) Discov. Med. 16:123-131.

Accordingly, the present invention preferably relates to the prevention or treatment of a disease selected from the group consisting of Aicardi-Goutiéres syndrome, familial chilblain lupus, spondyenchondromatosis, Systemic lupus erythematosus, in particular associated to a deleterious heterozygous mutation of the TREX gene, Sting-associated vasculopathy, Proteasome-associated auto-inflammatory syndrome (PRAAS), Singleton-Merten syndrome, Sjogren's syndrome, myositis, in particular polymyositis and dermatomyositis, psoriasis, systemic sclerosis, type I diabetes mellitus, autoimmune thyroid disease, rheumatoid arthritis, multiple sclerosis, and atherosclerosis.

### Individual

The individual according to the invention is preferably a mammal, more preferably a human. Preferably also, the individual according to the invention is a child or an infant.

Preferably, the individual according to the invention present with an abnormal or pathological level of IFN, in particular IFN-I, IFN-II and IFN-III, which is more preferably abnormally or pathologically elevated.

Preferably also the individual according to the invention presents an over-production or an excess of IFN, in particular IFN-I, IFN-II or IFN-III, or a high or elevated IFN level, in particular IFN-I, IFN-II or IFN-III level. The over-production or excess of IFN, in particular IFN-I, IFN-II or IFN-III, or high or elevated IFN level, in particular IFN-I, IFN-II or IFN-III level, can be either acquired, for instance as a consequence of a viral infection, or inherited, for instance as a genetic disorder.

In an embodiment of the invention, the individual according to the invention suffers from a chronic viral infection, in particular a chronic viral infection leading to an over-production of IFN, in particular IFN-I, IFN-II or IFN-III. Preferably, the individual according to the invention suffers from a chronic viral infection with virus a selected from the group consisting of the human immunodeficiency virus, influenza or dengue.

### Administration

Preferably, the CXCR4 receptor-binding compound according to the invention is administered in a prophylactically or therapeutically effective amount for preventing or treating a disorder associated to an over-production of IFN, notably for preventing or treating an interferonopathy or a disease as defined above. Preferably also, the CXCR4 receptor-binding compound according to the invention is administered in an amount suitable for decreasing IFN level in an individual.

The CXCR4 receptor-binding compound according to the invention can be administered by any route in the art, such as the intravenous, intramuscular, subcutaneous injection, oral, or topical routes.

### In Vitro Screening Method

Preferably, the in vitro screening method for identifying compounds for decreasing IFN level in an individual from candidate compounds, wherein the candidate compounds are CXCR4 receptor-binding compounds according to the invention, comprises the steps of:

contacting blood cells with a candidate compound;

determining the level of secretion of IFN by the contacted blood cells;

selecting the candidate compound which decreases the level of secretion of IFN with respect to the level of secretion of IFN before the blood cells have been contacted by the candidate compound, thereby identifying a compound for decreasing IFN level.

Preferably, the in vitro screening method according to the invention is performed by flow cytometry.

Blood cells according to the invention can be of any type known to one of skilled in the art. Preferably, blood cells according to the invention are peripheral blood mononuclear cells (PBMCs), more preferably plasmacytoid dendritic cells (pDCs), monocytes or NK cells.

Preferably, in vitro screening method for identifying compounds for decreasing IFN level in an individual from candidate compounds according to the invention, the CXRC4 receptor is expressed on the surface of cells, such as HEK cells.

The detectable CXCR4 receptor-biding compound according to the invention can be of any type known to one of skilled in the art. Preferably, the detectable CXCR4 receptor-biding compound according to the invention is an antibody, such as the 12G5 antibody, with a detectable label or a compound of formula (II) as defined above, in particular FFN102 and FFN511.

### In Silico Experiments

In silico methods for screening compound are well known to one of skilled in the art. In silico method according to the invention preferably refers to a method for identifying candidate compounds or designing compounds for decreasing IFN level in an individual via bioinformatics tools. In silico method according to the invention can be of any type such as docking, for instance using a software such as cDocker, structure-based, ligand-based, receptor dependent-quantitative structure-activity relationship (RD QSAR), quantitative structure-activity relationship (QSAR), quantitative structure-property relationship (QSPR), pharmacophore model and design de novo.

Preferably, the in silico method for screening compounds from candidate compounds, or for designing compounds, for decreasing IFN level in an individual according to the invention is an in silico docking experiments. For example, the in silico method for screening compounds from candidate compounds, or for designing compounds, for decreasing IFN level in an individual according to the invention can be performed by using the crystal structure of CXCR4 with a small ligand structurally related to CB, notably with IT1t, and then identifying the potential biding pocket on the CXCR4 extracellular domain.

Preferably, the designed compound or a candidate compound according to the invention interacts with at least 8 amino acids of a CXCR4 receptor represented by SEQ ID NO: 1, wherein the amino acids are selected from the group consisting of tryptophan 94, tryptophan 102, aspartic acid 97, aspartic acid 187, tyrosine 116, tyrosine 190, arginine 183, isoleucine 185, valine 112, cysteine 186 and glutamic acid 288.

The invention will be further described by the following non-limiting figures and Example.

### DESCRIPTION OF THE FIGURES

FIG. 1 shows the measure of IFN-α production (ng/ml) in the supernatants by Elisa by pDC pre-treated with histamine or with CB at the concentration of 10 μM and then stimulated with microvesicles (mock) alone or with HIV overnight.

The symbol 3 stars (***) represents P<0.001, the symbol 2 stars (**) represents P<0.01 and the symbol 1 stars (*) represents P<0.05.

FIG. 2 shows IFN-α quantified in the supernatants by ELISA by mouse MNC (multinucleated cells) obtained from the spleen using a homogenizer and purified using a 35% isotonic Percoll density gradient (Amersham Biosciences). Spleen MNC were depleted of RBC using red cell lysis buffer (8.3 mg/mL NH4Cl, 1 mg/mL KHCO3, and 3.72 μg/mL EDTA put in Mat and Med). Wild type (WT) (n=14) or H4RKO mice (n=10) spleen MNC were pre-incubated with CB (10 μM) and then cultured overnight with Influenza A virus.

FIG. 3 shows mRNA levels of TRAIL and IFN-(α,β) from purified pDC pre-incubated with histamine, CB, dopamine, serotonin and spermidine and stimulated overnight with HIV, measured by RT-qPCR and normalized to RPL13A. When not specify, data shown are representative of three independent experiments. P values (p) were determined using a two-tailed Student's t test. The symbol 3 stars (***) represents P<0.001, the symbol 2 stars (**) represents P<0.01 and the symbol 1 star (*) represents P<0.05.

FIGS. 4A-4C shows mRNA levels of IFN-α (FIG. 4A), IFN-β (FIG. 4B) and IFN-λ2/3 (FIG. 4C) from PBMC pre-incubated with histamine, and CB and stimulated overnight with Flu, measured by RT-qPCR and normalized to RPL13A. Data shown are representative of three independent experiments.

FIGS. 5A-5C show IFN-α (FIG. 5A), IFN-β (FIG. 5B) and IFN-λ2/3 (FIG. 5C) levels in BAL fluid measured by ELISA from 29S8 mice infected with X31 (800 TCID50). The symbol 3 stars (***) represents P<0.0001, the symbol 2 stars (**P) represents <0.001 and the symbol 1 star (*) represents P<0.01, by two-way ANOVA with Bonferroni post-tests.

FIG. 6 shows compound fixation on CXCR4 by flow cytometry from Jurkat cells incubated with CXCL12 (100 nM), HA (1 mM) or CB (1 mM) at 4° C. for 30 min before being stained with 12G5 antibody (anti-CXCR4).

FIG. 7 shows the TRAIL (first bar), IFN-α (second bar) and IFN-β (third bar) mRNA expression level in flu-exposed human PBMC in the presence of 10 μM/50 μM CB or 10 μM/50 μM IT1t relative to the mRNA expression level in control flu-exposed human PBMC (100%).

FIG. 8 shows the HIV-stimulated type I interferon production by human pDC in the absence (/) or the presence of clobenpropit (CB) or monoclonal antibody 12G5.

FIG. 9 shows the intracellular levels measured by flow cytometry of IFN-γ (whitebar), TNF-α (hatched bar) and CD107α (black bar) expressed by NK cells treated without or with IT1t, clobenpropit (CB) and spermine for 1 hour and then activated by K562 cells line.

FIG. 10 shows mRNA levels of IFN-γ from monocytes pre-incubated with CB, IT1t or chloroquine and then stimulated with HIV or lipopolysacharid (LPS) measured by RT-qPCR and normalized to RPL13A expression. Data shown are representative of two independent experiments.

FIG. 11 shows the average score for signs of arthritis of mice (murine model of collagen-induced arthritis) receiving once daily intraperitoneal injection of PBS (black square), prednisolone (triangle) and IT1t at 3 mg/kg (mpk) (circle), 10 mg/kg (mpk) (diamond-shape) and 30 mg/kg (mpk) (squared) for the days of the study.

FIG. 12 shows the average score for signs of arthritis of mice (murine model of collagen-induced arthritis) receiving once daily intraperitoneal injection of PBS (black square), prednisolone (triangle) and clobenpropit at 3 mg/kg (mpk) (circle), 10 mg/kg (mpk) (diamond-shape) and 30 mg/kg (mpk) (squared) for the days of the study.

FIG. 13 shows the average plasma concentration of IL-β of mice (murine model of collagen-induced arthritis)_treated by daily intraperitoneal injection of PBS (black bar), prednisolone (vertically hatched) and IT1t at 3 mg/kg (mpk) (hatched to the right), 10 mg/kg (mpk) (hatched to the left) and 30 mg/kg (mpk) (horizontally hatched), measured at day 14 (terminaison) The symbol one star (*) represents p≤0.05 vs PBS, the symbol two stars (**) represents p≤0.01 vs PBS, the symbol three stars (***) represents p≤0.001 vs PBS, the symbol four stars (****) represents p≤0.0001 vs PBS, the symbol five stars (*****) represents p<0.00001 vs PBS.

FIG. 14 shows the average plasma concentration of IL-β of mice (murine model of collagen-induced arthritis) treated by daily intraperitoneal injection of PBS (black bar), prednisolone (vertically hatched) and clobenpropit at 3 mg/kg (mpk) (hatched to the right), 10 mg/kg (mpk) (hatched to the left) and 30 mg/kg (mpk) (horizontally hatched). The symbol one star (*) represents p≤0.05 vs PBS, the symbol two stars (**) represents p≤0.01 vs PBS, the symbol three stars (***) represents p≤0.001 vs PBS, the symbol four stars (****) represents p≤0.0001 vs PBS, the symbol five stars (*****) represents p<0.00001 vs PBS.

FIG. 15 shows the average plasma concentration of IL-6 of mice (murine model of collagen-induced arthritis) treated by daily intraperitoneal injection of PBS (black bar), prednisolone (vertically hatched) and IT1t at 3 mg/kg (mpk) (hatched to the right), 10 mg/kg (mpk) (hatched to the left) and 30 mg/kg (mkp) (horizontally hatched). The symbol one star (*) represents p≤0.05 vs PBS, the symbol two stars (**) represents p≤0.01 vs PBS, the symbol three stars (***) represents p≤0.001 vs PBS, the symbol four stars (****) represents p≤0.0001 vs PBS, the symbol five stars (*****) represents p<0.00001 vs PBS.

FIG. 16 shows the average plasma concentration of IL-6 of mice (murine model of collagen-induced arthritis) treated by daily intraperitoneal injection of PBS (black bar), prednisolone (vertically hatched) and clobenpropit at 3 mg/kg (mpk) (hatched to the right), 10 mg/kg (mpk) (hatched to the left) and 30 mg/kg (mpk) (horizontally hatched). The symbol one star (*) represents p≤0.05 vs PBS, the symbol two stars (**) represents p≤0.01 vs PBS, the symbol three stars (***) represents p≤0.001 vs PBS, the symbol four stars (****) represents p≤0.0001 vs PBS, the symbol five stars (*****) represents p<0.00001 vs PBS.

FIG. 17 shows the average plasma concentration of TRAIL of mice (murine model of collagen-induced arthritis) treated by daily intraperitoneal injection of PBS (black bar), prednisolone (vertically hatched) and IT1t at 3 mg/kg (mpk) (hatched to the right), 10 mg/kg (mpk) (hatched to the left) and 30 mg/kg (mpk) (horizontally hatched). The symbol one star (*) represents p≤0.05 vs PBS, the symbol two stars (**) represents p≤0.01 vs PBS, the symbol three stars (***) represents p≤0.001 vs PBS, the symbol four stars (****) represents p≤0.0001 vs PBS, the symbol five stars (*****) represents p<0.00001 vs PBS.

FIG. 18 shows the average plasma concentration of TRAIL of mice (murine model of collagen-induced arthritis) treated by daily intraperitoneal injection of PBS (black bar), prednisolone (vertically hatched) and clobenpropit at 3 mg/kg (mpk) (hatched to the right), 10 mg/kg (mpk) (hatched to the left) and 30 mg/kg (mpk) (horizontally hatched). The symbol one star (*) represents p≤0.05 vs PBS, the symbol two stars (**) represents p≤0.01 vs PBS, the symbol three stars (***) represents p≤0.001 vs PBS, the symbol four stars (****) represents p≤0.0001 vs PBS, the symbol five stars (*****) represents p<0.00001 vs PBS.

FIG. 19 shows the body weight in gram (g) of mouse (Pristane-Induced Systemic Lupus Erythematosus (SLE) Model in Balb/c Mice) treated with vehicle (PBS) (diamond-shape), positive control (prednisolone) (black square with dashed line) and clobenpropit at 3 mg/kg ((triangle), 10 mg/kg (black square with dotted line) and 30 mg/kg (star symbol).

FIG. 20 shows the body weight in gram (g) of mouse (Pristane-Induced Systemic Lupus Erythematosus (SLE) Model in Balb/c Mice) treated with vehicle (PBS) (diamond-shape), positive control (prednisolone) (black square with dashed line) and IT1t at 3 mg/kg ((circle), 10 mg/kg (black square with dotted line) and 30 mg/kg (black line).

FIG. 21 shows the level of dsDNA level in a pristane-Induced systemic lupus erythematosus (SLE) model in Balb/c mice treated with vehicle (black bar), prednisolone (dotted bar), clobenpropit at 3 mg/Kg (bar hatched to the right), 10 mg/Kg (bar with dashes), 30 mg/Kg (tile bar), IT1t at 3 mg/Kg (black bar with white tiles), 10 mg/Kg (bar with diamond shape), 30 mg/Kg (vertically hatched bar).

### EXAMPLE I: INHIBITION OF INTERFERON PRODUCTION BY VIRUS-STIMULATED PLASMACYTOID DENDRITIC CELLS

A. Materials and methods

1. Blood Samples, Isolation and Culture of Blood Leukocytes.

Blood was obtained from healthy HIV-1-seronegative blood bank donors. Experimental procedures with human blood were done according to the European Union guidelines and the Declaration of Helsinki. In vitro experiments were performed using human peripheral blood mononuclear cells (PBMC) isolated by density centrifugation from peripheral blood leukocyte separation medium (Cambrex, Gaithersburg, Md.). pDC were purified by negative selection with the Human plasmacytoid DC enrichment kit (StemCell Technologies). Cells were cultured in RPMI 1640 (Invitrogen, Gaithersburg, Md.) containing 10% fetal bovine serum (Hyclone, Logan, Utah). After purification, the purity obtained was higher than 91% for pDC.

2. Viral Stimulation and Infection.

PBMC were seeded at 1.10⁶/1 mL or purified pDC were seeded at 5.10⁴/100 μl and then stimulated with the following viruses: inactivated AT-2 HIV-1_{MN} (CXCR4 co-receptor specific) or AT-2 HIV-1_{ADA} (CCR5 co-receptor specific) at 60 ng/mL p24^{CA} equivalent (provided by J. D. Lifson (SAIC-NCI, Frederick, Md.)), Infectious human Influenza A/PR/8/34 virus (Flu), titer 1:8192 at dilution 1:1000 or DENV-2 16681 at MOI 10. Infectious HIV-1_{MN} [tissue culture 50% infective dose (TCID50)=106] and HIV-1_{ADA} (TCID50=1,000) were used at the same concentration. Purified pDC were pre-treated with amino compounds for 1 hour, following overnight stimulation with virus. Supernatants were collected for cytokine detection. Microvesicles isolated from uninfected cell cultures matched to the culture to produce the virus were used as negative control (Mock).

3. Chemical Compounds.

Histamine dihydrochloride, clobenpropit dihydrobromide, dopamine, serotonin and spermidine (Sigma-Aldrich, MO, USA) were diluted in pure water and IT1T (R&D system/Tocris) was diluted in DMSO. The compounds were added in pDC culture at 10 μM (or other if specified) 1 hour before stimulation or not of the different viruses. For histamine, X-vivo culture media (Lonza) was used in order to avoid histaminases.

Fluorescent compounds FFN-511 and FC—CO₂⁻ were synthetized similarly to the procedure described in in Gubernator et al (2009) Science, 324: 1441-1444 and Lee et al (2010) Journal of the American Chemical Society, 132: 8828-8830. Cells were pre-incubated 1 hour with AMD (20 μM) (Sigma-Aldrich, MO, USA) prior to CB or histamine incubation. pDC were cultured in the presence of 5 mM of the oligodinucleotide A151 (TTAGGG) ODN (Integrated DNA Technologies, Coralville, Iowa). The histamine receptors antagonists (pyrilamine/PYR for H1R, cimetidine/CIM for H2R, thioperamide/THIO for H3R and JNJ7777120/JNJ and A943931 for H4R) (Sigma-Aldrich, MO, USA) were used at 10 μM.

4. H4R and CXCR4 Knockout Experiments.

pDCs were seeded at 10⁵ cells/mL in 96-well plates and incubated at 37° C. H4R and CXCR4 Small interfering RNA (siRNA) (Smart Pool, Dharmarcon) was diluted in DOTAP (Roche Applied Sciences). The mix was gently mixed and incubated at room temperature during 15 minutes. After incubation, the mix was added to cells in culture at a final concentration of 160 nM. Finally, cells were incubated at 37° C. for 24 hours before adding the different viruses overnight. Control was performed using a siRNA control.

5. Flow Cytometry.

Cultured cells were incubated for 20 min at 4° C. with appropriate antibodies Phycoerythrin (PE)-conjugated TRAIL clone RIK-2 (BD Bioscience, San Jose, Calif.), APC-conjugated BDCA-4, FITC-CD123 (Miltenyi, Bergisch Gladbach, Germany), FITC-HLADR, PercP-cy5.5-CCR7, APC-CD40, BV421-CD80, FITC-CD86, PE-CXCR4 clone 12G5 (Biolegend, San Diego, Calif.) or with appropriate isotype-matched control antibodies (5 μg/mL each) in PBS containing 2% mouse serum (Sigma, Saint Louis, Mo.) and FC-receptor blockers (BD Biosciences, San Jose, Calif.). Flow cytometry analysis was performed on a flow cytometry Canto II or LSR II flow cytometer using flow cytometry Diva software (BD Biosciences, San Jose, Calif.). FlowJo software (Treestar, Ashland, Oreg.) was used to analyze data.

6. Cytokine Detection.

pDC's supernatants were tested for multispecies soluble IFN-α by ELISA (PBL Assay Science, NJ, USA) according to the manufacturer's instructions.

7. RT-qPCR Analyses.

Total RNA was extracted using RNeasy Micro kit and was submitted to DNase treatment (Qiagen), following manufacturer's instructions. RNA concentration and purity were evaluated by spectrophotometry (Biophotometer, Eppendorf). Five hundred ng of RNA were reverse-transcribed using PrimeScript RT Reagent Kit (Perfect Real Time, Takara) in a 10 μl reaction. Real-time PCR reactions were performed in duplicates using Takyon ROX SYBR MasterMix blue dTTP (Eurogentec) on a 7900HT Fast Real-Time PCR System (Applied Biosystems). Transcripts were quantified using the following program: 3 min at 95° C. followed by 35 cycles of 15 s at 95° C., 20 s at 60° C. and 20 s at 72° C. Values for each transcript were normalized to expression levels of RPL13A (60S ribosomal protein L13a) using the 2-ΔΔCt method. Primers used for quantification of transcripts by real time quantitative PCR are indicated below:

| | Forward primer | Reverse Primer | Size |
|---|---|---|---|
| Gene | sequence (5′→3′) | sequence (5′→3′) | (bp) |
| RPL13A | CCTGGAGGAGAAGAG | TTGAGGACCTCTGTG | 126 |
| | GAAAGAGA | TATTTGTCAA | |
| | (SEQ ID NO: 2) | (SEQ ID NO: 3) | |
| TRAIL | GCTGAAGCAGATGCA | TGACGGAGTTGCCAC | 135 |
| | GGACAA | TTGACT | |
| | (SEQ ID NO: 4) | (SEQ ID NO: 5) | |
| IFN- | CCAGTTCCAGAAGGC | TCCTCCTGCATCACA | 174 |
| a1/13¹ | TCCAG | CAGGC | |
| | (SEQ ID NO: 6) | (SEQ ID NO: 7) | |
| IFN- | CCCACAGCCTGGGTA | CAGCAGATGAGTCCT | 210 |
| a4/10²v | ATAGGA | CTGTGC | |
| | (SEQ ID NO: 8) | (SEQ ID NO: 9) | |
| IFN-β | TGCATTACCTGAAGG | AGCAATTGTCCAGTC | 152 |
| | CCAAGG | CCAGTG | |
| | (SEQ ID NO: 10) | (SEQ ID NO: 11) | |
| IFN-λ1 | GGACGCCTTGGAAGA | CTGGTCTAGGACGTC | 17 |
| | GTCAC | CTCCA | |
| | (SEQ ID NO: 12) | (SEQ ID NO: 13) | |
| IFN- | GGGCCTGTATCCAGC | GAGGAGGCGGAAGAG | 16 |
| λ2/3³ | CTCAG | GTTGA | |
| | (SEQ ID NO: 14) | (SEQ ID NO: 15) | |
| IFN-γ | GGCAGCCAACCTAAG | CAGGGTCACCTGACA | 17 |
| | CAAGAT | CATTCA | |
| | (SEQ ID NO: 16) | (SEQ ID NO: 17) | |
| IL6 | TAACCACCCCTGACC | ATTTGCCGAAGAGCC | 14 |
| | CAACC | CTCAG | |
| | (SEQ ID NO: 18) | (SEQ ID NO: 19) | |
| IL8 | AAGGGCCAAGAGAAT | ACTAGGGTTGCCAGA | 165 |
| | ATCCGAA | TTTAACA | |
| | (SEQ ID NO: 20) | (SEQ ID NO: 21) | |
| IL10 | AAGGGCCAAGAGAAT | GCTGGCCACAGCTTT | 146 |
| | ATCCGAA | CAAGA | |
| | (SEQ ID NO: 22) | (SEQ ID NO: 23) | |
| IL15 | AAGAAGAGCTGGCTA | TCATGTTCCATGCTG | 142 |
| | TGGCA | CTGAC | |
| | (SEQ ID NO: 24) | (SEQ ID NO: 25) | |
| ISG5 | AGGACAGGAAGCTGA | AGTGGGTGTTTCCTG | 19 |
| | AGGAG | CAAGG | |
| | (SEQ ID NO: 26) | (SEQ ID NO: 27) | |
| CXCL10 | CGCTGTACCTGCATC | GCAATGATCTCAACA | 107 |
| | AGCAT | CGTGGAC | |
| | (SEQ ID NO: 28) | (SEQ ID NO: 29) | |
| iNOS | CAGCGGGATGACTTT | AGGCAAGATTTGGAC | 75 |
| | CCAA | CTGCA | |
| | (SEQ ID NO: 30) | (SEQ ID NO: 31) | |
| CXCR4 | GCATGACGGACAAGT | AAAGTACCAGTTTGC | 101 |
| | ACAGGCT | CACGGC | |
| | (SEQ ID NO: 32) | (SEQ ID NO: 33) | |
| H4R | ACACGCTGTTCGAAT | TCGATCATAGCTGAT | 113 |
| | GGGAT | GAGGACAA | |
| | (SEQ ID NO: 34) | (SEQ ID NO: 35) | |

¹Primers amplify both IFN-a1 and IFN-a13 transcripts
²Primers amplify both IFN-a4 and IFN-a10 transcripts
³Primers amplify both IFN-λ2 (IL-28A) and IFN-λ3 (IL-28B) transcripts.

8. In Vivo Treatment of Mice.

12 weeks old 129S8 mice (Jackson Laboratory), bred at the MRC-National Institute for Medical Research (NIMR) under specific pathogen-free conditions, were treated with Clobenpropit dihydrobromide (Sigma-Aldrich, C209) (450 μg/30 μL/mouse), Histamine dihydrochloride (Sigma-Aldrich, H7250) (450 μg/30 μL/mouse) or Vehicle Control (PBS) (30 μL/mouse) 18 hours prior to infection. Mice were infected with Influenza A virus strain X31 (H3N2) (a kind gift from Dr. J. Skehel, MRC-NIMR) at 800 TCID/30 μL. X31 was grown in the allantoic cavity of 10 day-embryonated hen's eggs and was free of bacterial, mycoplasma, and endotoxin contamination, stored at −70° C. and titrated on MDCK cells, according to the Spearman-Karber method. All mice were treated and infected intranasally (i.n) under light isoflurane-induced anaesthesia. At 3 days post infection mice were euthanized and bronchioalveolar lavage (BAL) fluid was collected. BAL samples were centrifuged at 1,300 rpm, 5 min at 4° C. and supernatant collected. Samples were then analysed for concentrations of IFNα, (eBioscience) IFNβ (Biolegend UK) and IFNλ (R&D) by ELISA as per the manufacturer's instructions.

9. Three-Dimensional Microscopy.

In some cases, purified pDC cells cultured overnight in presence of HIV-1 and with the different compounds (CB, FFN-511 and FC—CO₂⁻). pDC (1×10⁵ cells/slide) were plated on collagen-coated slides and fixed with paraformaldehyde 4%. Cells were then incubated with anti-CXCR4 antibody (Biolegend, San Diego, Calif.) in saturation buffer PBS-BSA 0.5% for membrane staining or in permeabilizing buffer containing 1% saponin with monoclonal antibody anti-TRAIL (Biolegend, San Diego, Calif., USA). CXCR4 was revealed by a donkey anti-mouse IgG-AF647 (Molecular Probes, OR, USA) and TRAIL was revealed by a Donkey anti-mouse IgG-Cyanine 3 (Jackson ImmunoResearch, West Grove, Pa.). Nucleus was stained using DAPI (Molecular Probes, Paisley, UK). Slides were mounted with Fluoromount-G (eBioscience, CA, USA) and scanned with a Nikon Eclipse 90i Upright microscope (Nikon Instruments Europe, Badhoevedorp, The Netherlands) using a 100× Plan Apo VC piezo objective (NA 1.4) and Chroma bloc filters (ET-DAPI, ET-GFP) and were subsequently deconvoluted with a Meinel algorithm and 8 iterations and analyzed using Metamorph® (MDS Analytical Technologies, Winnersh, UK). TRAIL/DAPI/Overlay/Confocal plane: Representative 2D focal plan. Overlay with bright: Bright. Reconvolution overlays: 2D projections of the maximum intensity pixels along the Z-axis.

In other cases, cells were cultured overnight in media alone then were washed in ice-cold PBS-BSA 0.2% and stained with CXCR4 antibody (R&D Systems) for 1 hour at 4° C. Cells were then washed with PBS-BSA 0.2% and stimulated with CB for 1 hour at 4° C. Data are expressed as the mean percentage ±SEM mean channel fluorescence intensity (MFI) values for residual surface expression and intracellular staining. After staining in suspension, pDC (1×10⁵ cells/slide) were spun for 10 min at 400 rpm with a Shandon Cytospin® Cytocentrifuge (Thermo Scientific, St-Herblain, France) and fixed with paraformaldehyde 4%. Cells were then stained with secondary antibody anti-mouse-AF647 (Molecular Probes, OR, USA) either at the membrane or after permeabilization with Triton 0.2%. Finally, slides were washed in PBS, counterstained with Hoechst 33342 and mounted in Fluoromount-G medium. Images were digitally acquired with a Zeiss LSM 710 confocal Microscope using 63×PL APO O.N.=1.4/oil objective.

All analyses were performed using the ImageJ software (NIH, Bethesda, Md., USA).

10. Image Quantification.

7 pictures were taken from each slide for each Z section framing the nucleus. Quantification of the colocalization in the cytoplasm of purified pDC using Mander's Coefficient was obtained after analyze by JACoP pluging in ImageJ.

11. CXCR4 Internalization.

Interaction with CXCR4. CB and histamine binding to CXCR4 was assessed by flow cytometry analysis (FACSCantoll; Becton Dickinson) of Jurkat cells using anti-human CXCR4 antibodies. Briefly, Jurkat cells were pre-incubated with CB, histamine (1,000 μM) or buffer for 30 min at 4° C. in FACS buffer (PBS-1% FCS). After incubation, cells were washed with FACS buffer by centrifugation, then stained with PE-labeled anti-human CXCR4 antibodies 12G5 (Pharmingen) for 30 min at 4° C. After being washed, the cells were fixed with 4% paraformaldehyde in FACS buffer for 5 min at 4° C. CXCR4 staining was quantified by flow cytometric analysis (10,000 cells per sample) on a cytometer (FACSCantoll, Becton-Dickinson). Data were processed using FACSDiva software (Becton Dickinson). All values represent mean fluorescence intensities of cells relative to CXCR4 levels in buffer-treated cells (100%) from a triplicate experiment ±SD. Statistical calculations were performed with a two-tailed paired Student's t-test using GraphPad Prism Version 5.03. p<0.05 was considered significant.

Internalization of CXCR4. Internalization of CXCR4 was assessed by flow cytometry analysis of Jurkat cells using an anti-human CXCR4 antibody. Briefly, Jurkat cells were pre-incubated with CB (10 μM), CXCL12 (250 nM) or buffer for 30 min at 37° C. in serum-free medium. After incubation, cells were washed with FACS buffer by centrifugation, then sequentially stained with PE-labeled anti-human CXCR4 antibody (1D9, BD Pharmingen) for 30 min at 4° C. After being washed, the cells were fixed with 4% paraformaldehyde in FACS buffer for 5 min at 4° C. CXCR4 expression was quantified by flow cytometric analysis (10,000 cells per sample) on a cytometer. Data were processed using FACSDiva software (Becton Dickinson). All values represent mean fluorescence intensities of cells relative to CXCR4 expression in buffer-treated cells (100%) from a triplicate experiment±SD. Statistical calculations were performed with a two-tailed paired Student's t-test using GraphPad Prism Version 5.03. p<0.05 was considered significant.

12. Molecular Modelling of CXCR4 with Various Ligands.

The molecular docking program cDOCKER was used for automated molecular docking simulations and various scoring function were used to rank poses: Jain, cDocker Interaction optimized, Ludi. PDB files were cleaned using the prepare protein protocol of Discovery Studio 4.1, membrane was added according to Im. W algorithm. Ligands and their conformer were prepared using prepare ligand protocol after conformation generation. Complexes were selected on the basis of criteria of interacting energy combined with geometrical matching quality as well as compromise of scoring function. Figures were generated with Discovery studio 4.1 graphics system. The 2D representations of molecular structures interaction of Discovery Studio was used for delineation of the detailed interactions between ligands and CXCR4 (PDB code: 3ODU). An interaction was considered a hydrophobic interaction if the Van der Walls fraction was 0.7 and was considered a hydrogen bond if it was between a listed donor and acceptor and the angles and distances formed by the atoms surrounding the hydrogen bond lay within the default criteria. RMSD were calculated using Discovery studio 4.1 and with IT1t in CXCR4/IT1t co-crystal as reference (PDB code 3ODU).

13. Statistical Analysis.

P values (P) were determined using a two-tailed Student's t test. P<0.05 was considered statistically significant. *=P<0.05; **=P<0.01 and ***=P<0.001. Univariate distributions of flow cytometry data were performed by probability binning, in 300 bins using FlowJo software.

For mice data, data shown as the means±s.e.m. Sample sizes were designed to give statistical power, while minimizing animal use. Data sets were analysed by two-way ANOVA with Bonferroni post-tests (cytokine concentration time courses). GraphPad Prism 5 (GraphPad Software, San Diego, Calif.) was used for data analysis and preparation of all graphs. P-values less than 0.01 were considered to be statistically significant.

B. Results

1. Histamine and Clobenpropit Inhibit HIV-Induced pDC Activation.

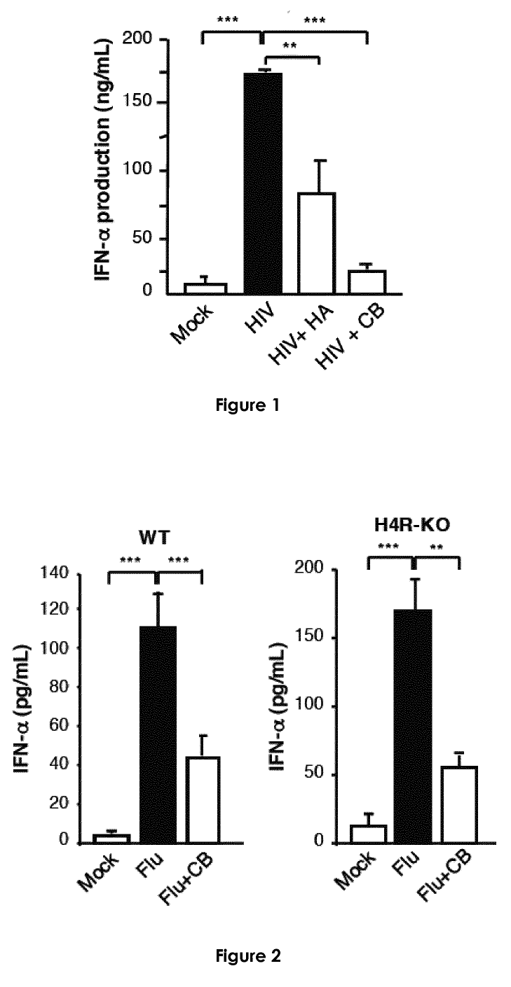

The effect of histamine on pDC activation by HIV-1 has been examined. A dose range analysis indicated that histamine was active at 10 μM on purified pDC without obvious toxicity. The effect of the H4R agonist clobenpropit (CB) has been tested. CB showed a stronger inhibitory effect than histamine, and reduced levels of IFN-α secreted following HIV stimulation by approximately 90% (FIG. 1). CB had no cytotoxic effect at the concentration of 10 μM. Next IFN-α production kinetics was assessed and showed that CB inhibited IFN-α production by HIV-stimulated pDC after 9 h of incubation. The CB inhibitory effect was compared to a TLR-7 antagonist, A151 and it could be showed that both molecules were similarly active. Relative TRAIL mRNA expression levels were assessed by RT-qPCR and confirmed these results. CB also strongly inhibited IFN-α production and membrane TRAIL expression by pDC cultured with Flu and Dengue, demonstrating that CB effect was not restricted to HIV.

Then endogenous and synthetic amines was tested in a range from 5.10⁻⁷ to 10⁻³ M on type I IFN production and TRAIL expression on human Flu-activated primary PBMC. Furthermore, cell viability under the several concentrations of amines was simultaneously studied. It showed that amines optimal effects were observed between 10⁻⁵ and 5.10⁻⁵ M for the vast majority of the molecules. Higher concentrations induced high levels of cell death (Table 1).

TABLE 1
Summary of the EC50, the TC50 and the therapeutic index of
histamine (HA), clobenpropit (CB), serotonine (5-HT) and
IT1†.

| | | | Therapeutic index |
|---|---|---|---|
| Compounds | EC50 (μM) | TC50 (μM) | (Ratio TC50/EC50) |
| Histamine | 1.4 ± 1 | <2 mM | <1500 |
| Clobenpropit | 24 ± 2 | 417 ± 6 | 17.4 |
| Serotonin | 1.3 ± 0.5/113 ± 3 | 1500 ± 54 | 1150/13.3 |
| IT1† | 7.1 ± 1 | 237.9 ± 21 | 33.5 |

2. The Histamine Receptors are not Involved in Inhibition of pDC.

It has been tested whether the activity of CB was dependent on histamine receptors. CB in the presence of different histamine receptor antagonists was evaluated (pyrilamine/PYR for H1R, cimetidine/CIM for H2R, thioperamide/THIO for H3R and JNJ7777120/JNJ or A943931 compounds for H4R at 10 μM on Flu-stimulated pDC. It has been found that none of these antagonists reversed inhibition of IFN-α production triggered by CB. To confirm these results, CB and histamine were analyzed on viral activation of pDC isolated from wild type (WT) or H4R knock out (KO) mice. In these experiments, Flu was used to stimulate cells.

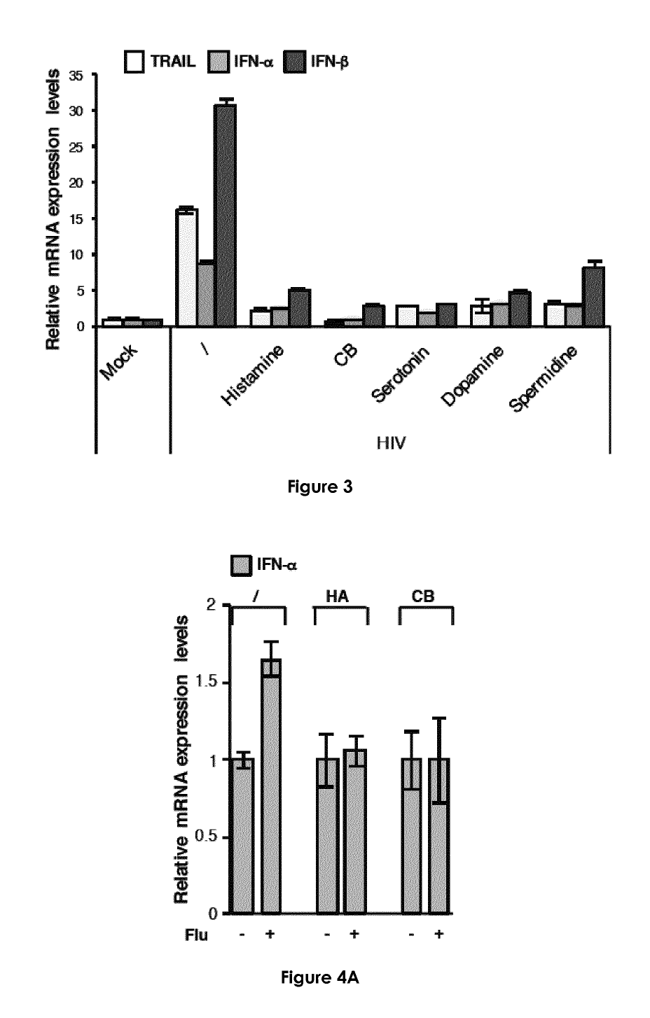

Indeed, HIV is unable to induce type I IFN or TRAIL expressions in mouse pDC because mouse pDC do not express the HIV coreceptor CD4, which is essential for pDC recognition and activation. CB inhibited IFN-α production by Flu-stimulated pDC from both wild type and H4R KO mice (FIG. 2). Next, H4R was silenced in human primary pDC by siRNA, and then the effect of histamine and CB was determined. We found that H4R knock down did not block histamine nor CB inhibitory activity on IFN-α, IFN-β and TRAIL productions by HIV-stimulated pDC. Thus, H4R is not implicated in the model of pDC modulation by histamine or CB, suggesting an alternative mechanism. Thus it has been examined whether amines in general display an inhibitory effect on pDC activation and natural amines dopamine, serotonin and spermidine were analyzed. All amines inhibited HIV-mediated membrane TRAIL and HLADR, as well as migration and maturation markers as CCR7, CD40, CD86 and CD80 expression, and also TRAIL, IFN-α/β mRNA by HIV-stimulated pDC (FIG. 3). Notably, none of these molecules were cytotoxic at concentration used. Different amines alone on human primary pDC culture were also tested. As positive control HIV was used to stimulate cytokine production by pDC. Membrane TRAIL and HLADR, as well as migration and maturation markers as CCR7, CD40, CD86 and CD80 expression were not affected by the amines. IFN-α, IFN-β and TRAIL mRNA expressions were quantified by RT-PCR and showed that none of the amines tested had an effect alone on type I IFN production.

3. Histamine and Clobenpropit Inhibit Flu-Induced Production of Interferon in PBMC and 129S8 Mice.

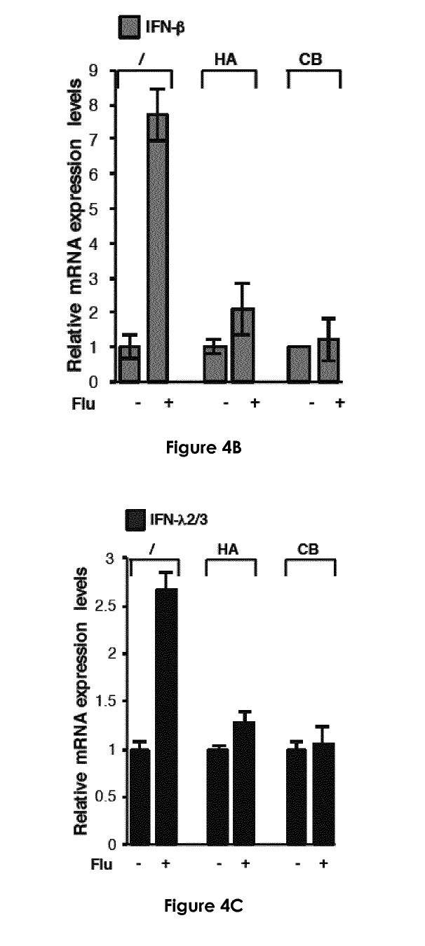

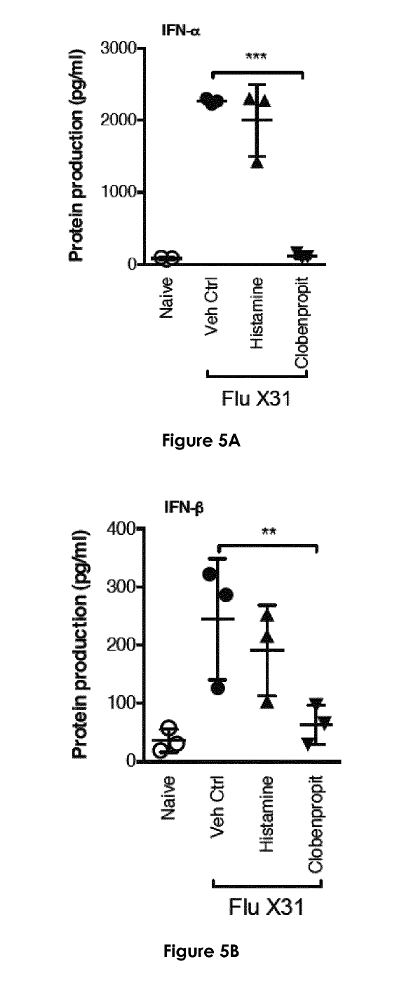

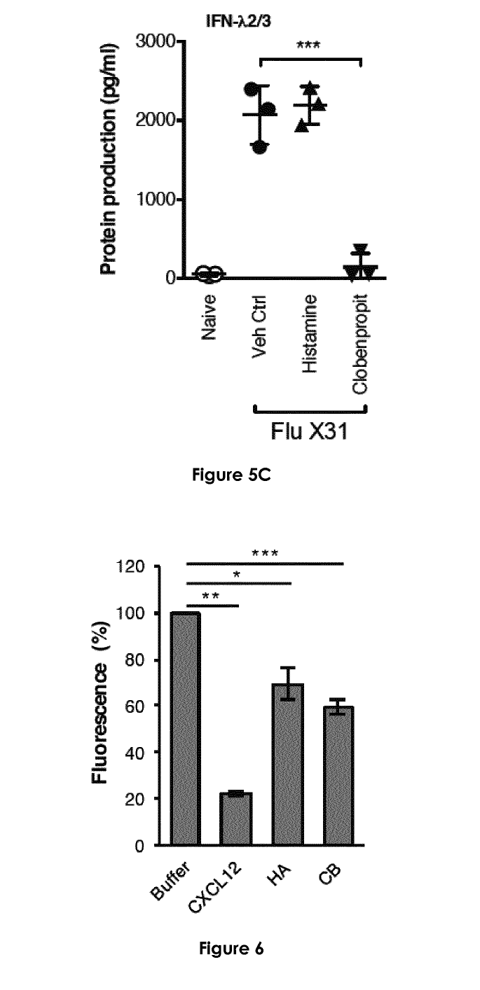

CB and Histamine effects on flu-exposed human PBMC were first tested. IFN-α/β and IFN-λ2/3 mRNA levels were significantly reduced when cells were pre-treated with histamine or CB before Flu exposure, validating that amines had inhibitory activity in a mixed culture system containing various immune cell populations (FIGS. 4A-4C). Next it has been investigated whether amines exhibit inhibitory activity on antiviral cytokine responses in vivo. We determined how histamine and CB affect IFN production in 12-week-old 129S8 mice infected with the X31 Flu strain or inoculated with vehicle control. At day 3 of influenza infection, mice pre-treated with CB showed a strong reduction of IFN-α, IFN-β and IFN-λ2/3 protein production in bronchioalveolar lavage (BAL) fluid compared to untreated Flu-infected mice (FIGS. 5A-5C). When mice were treated with histamine prior to influenza infection, a trend towards IFN reduction that was not statistically significant was noticed. This result may be explained by the fact that histamine is a natural amine, and therefore degraded by histaminase found in serum.

4. The Ammonium Group (NH3+) is Important to Inhibit pDC Activation.

To further study the role of amines on pDC activation, FFN-511, a fluorescent amine mimetic of serotonin was synthetized. This compound contains an ammonium group (NH₃⁺) and a fluorescent coumarin core allowing microscopy and flow cytometry analysis. FFN-511 (at 50 μM), strongly reduced IFN type I production by HIV-stimulated pDC without any obvious cytotoxic effect. To further investigate the role of the NH3⁺ function, a negatively charged analog of FFN-511 was synthesized, FC—CO₂⁻ in which the ammonium group (NH₃⁺) was replaced by a carboxylic (CO₂⁻) moiety. The effect of the amine FFN-511 and its analogue FC— CO₂⁻ were examined on a panel of activation markers, using an RT-qPCR profiling assay. A panel of genes that are usually activated after viral exposure were selected: TRAIL, IFNs (IFN-α, IFN-β, IFN-γ, IFN-λ1 and IFN-λ2/3), interleukins (IL6, IL8, IL10 and IL15), chemokines (CXCL10), inducible nitric oxide synthase (iNOS) and an early ISG (ISG56). Values for each transcript were normalized to expression level of ribosomal protein L13a (RPL13A). All virus-induced genes were induced in pDC by HIV-1 and their transcription was dramatically inhibited by CB and FFN-511 but not by FC— CO₂⁻. However, neither CB nor FFN-511 affected iNOS or IL-15 gene expression, suggesting a specific inhibition of virus-induced gene expression rather than a global effect on cellular gene transcription.

5. Synthetic and Natural Amines Inhibit TRAIL Relocalization at the Membrane of HIV-Stimulated pDC.

Viral exposure results in the relocalization of TRAIL from the cytoplasm to the cell membrane. Thus, it has been examined whether amines affect this process using 3 dimensions (3D) microscopy. As expected, TRAIL was mostly localized in the cytosol in non-activated pDC, but became detectable at the plasma membrane upon HIV stimulation. CB and FFN-511 significantly inhibited TRAIL localization to the cell membrane in HIV-stimulated pDC whereas FC—CO₂⁻ did not. Image quantification of membrane TRAIL was performed and validated by flow cytometry results. Therefore, upon HIV-1 exposure, amines prevent the surface access of an intracellular pool of TRAIL thus inhibiting the pro-killer activity of HIV-activated pDC10.

6. The Chemokine Receptor CXCR4 is Required for Amine Inhibitory Effect on pDC.

It has been found that HA and CB inhibited binding of the CXCR4 antibody 12G5 on T cells (FIG. 6), thus confirming a direct interaction of the amines with CXCR4. Furthermore, intracellular and/or extracellular levels of CXCR4 were assessed by staining permeabilized and non-permeabilized pDC with receptor specific antibody. When cells were incubated with CB, most of CXCR4 was detected intracellularly, compared to untreated cells. In addition, internalization of CXCR4 was also assessed by flow cytometry analysis of Jurkat T cells using an anti-human CXCR4 antibody clone 1D931. To visualize the interaction between CXCR4 and amines, the fluorescent properties of FFN-511 were used. Confocal microscopy of pDC demonstrated a strong degree of co-localization between FFN-511 and CXCR4. As it has been shown that the compounds could internalize CXCR4, the effect of CXCR4 natural ligand, CXCL12, on pDC activation was studied. Purified pDC were pre-incubated at different time points (15 min, 30 min, 60 min) of CXCL12 (62.5 nM). It has been found that whatever the time of pre-incubation, CXCL12 did not reduce IFN-α/β mRNA expressions by HIV stimulated pDC. To confirm these surprising results CXCL12 was used at the concentration used for amines (10 μM) and even at this concentration CXCL12 did not inhibit type I IFN mRNA expression. Thus, the inventors demonstrated that CXCL12 did not act as amines and was not able to inhibit viral activation of human pDC. Then CXCR4 was silenced in pDC using small interfering RNA (siRNA). CXCR4 gene silencing suppressed the inhibitory effect of histamine or CB on type I IFN and TRAIL, in pDC stimulated by CXCR4-tropic HIV-1. It should be noticed that CXCR4 is not required for pDC activation by HIV-1. To generalize the findings, it was verified that CXCR4 silencing also blocked CB inhibitory effect on pDC activated by a CCR5-tropic (R5) HIV-1 and Flu. Thus, amines inhibit virus sensing in pDC by engagement of CXCR4.

7. Identification of a Binding Pocket for Amines on CXCR4 Extracellular Domain.

To better understand the molecular interaction between amines and CXCR4, in silico docking experiments were performed. IT1t was used as an internal control to validate the molecular modeling protocol. Thus, IT1t and the compounds were docked in the IT1T binding pocket of CXCR4. First it has been confirmed that IT1t was replaced properly compared to the crystal structure. Indeed, RMSDs of IT1t heavy atoms resolved in crystal structure (PDB code 3ODU) and IT1t docking poses after scoring are around 1 Å (equivalent to the variation observed when comparing IT1t in 3ODU co-crystal with other co-crystallized structures (PDB codes 3OE6-3OE8-3OE9)) (Table 2).

TABLE 2
Validation of docking protocol. Scoring of IT1† poses
after docking in CXCR4 (PDB code: 3ODU) using cDocker.
Poses were ranked depending on their scores calculated
either with Jain, cDocker Interaction Optimized or Ludi
as scoring function. RMSD between each top poses and
crystallized IT1† as reference was calculated in Å.

| | | | | X-ray | |
|---|---|---|---|---|---|
| | | | | IT1† | |
| | Scoring Function | | | (PDB code 3ODU) vs | |
| | | cDocker | | IT1† in other structures | |
| | Jain | Interaction | Ludi | (PDB codes: | |
| | RMSD | Optimized | RMSD | 3OE6-3OE8-3OE9) | |
| Top | heavy | RMSD heavy | heavy | PDB | RMSD heavy |
| pose | atoms | atoms | atoms | code | atoms |
| #1 | 1.0603 | 1.0603 | 1.0603 | 3OE6 | 0.3887 |
| #2 | 0.7775 | 0.9667 | 0.7775 | 3OE8 | 0.6986 |
| #3 | 0.9667 | 3.8033 | 0.9667 | 3OE9 | 0.6739 |

RMSD: Root-mean-square deviation.

Various conformers of histamine, CB, and FFN-511 were docked into CXCR4 and the complex was minimized to establish the optimal model.

The 2D representation was used for delineation of the detailed interactions between ligands and CXCR4. With this model, all tested ligands were potentially interacting with CXCR4 in the same extracellular pocket than IT1t and with key residues previously characterized (Table 3).

TABLE 3
Residues involved in ligand binding in IT1†
IT1T Binding pocket

| | IT1T | CB | AMD3100 |
|---|---|---|---|
| W94 | X | X | |
| D97 | X | X | |
| W102 | X | X | |
| V112 | X | | |
| Y116 | X | X | |
| R183 | X | X | |
| I185 | X | X | |
| C186 | X | X | |
| D187 | X | X | |
| Y190 | | X | |
| E288 | X | X | X |

Poses were scored and compounds were classified depending of their properties. A high score indicates a strong interaction with various residues inside the pocket. As expected, FC—CO₂⁻ showed the lowest score, indicating a weak interaction between the compound and the binding site of CXCR4. Moreover, in silico scores of compounds directly correlated with their experimental potency. Since the putative binding site for amines in CXCR4 overlaps the binding pocket of IT1t, it has then been studied whether IT1t could inhibit type I IFN and TRAIL production by virus-exposed pDC.

8. IT1t Inhibit HIV or Flu-Induced Expression of Interferon in Human pDC or PBMC

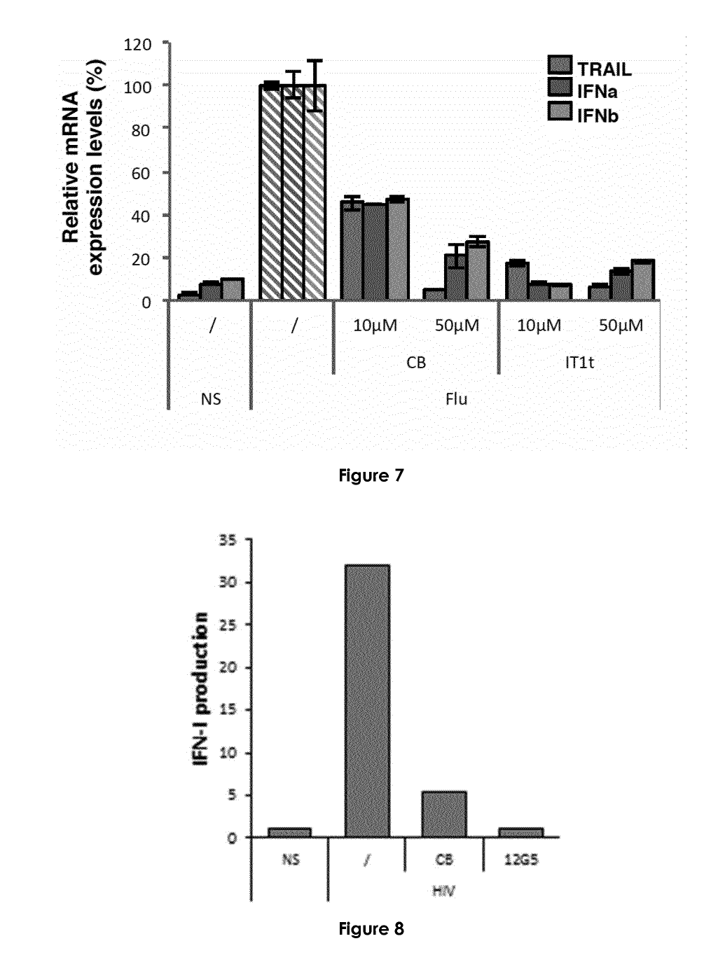

Human pDC were cultured with IT1t for 1 hour followed by exposure to HIV X4. It has been found that IT1t inhibited IFN-α, IFN-β and TRAIL expression by HIV-stimulated pDC. By comparing CB and IT1t effect on cytokine production of activated pDC, a stronger effect of IT1t than CB was shown. Similar results were obtained for flu-exposed human PBMC (FIG. 7).

IT1t was not toxic at the efficient concentrations, but showed some toxicity at higher concentrations, probably due to the DMSO in which it was diluted. Furthermore, to demonstrate that IT1t activity was mediated through CXCR4 engagement, CXCR4 RNA silencing in human pDC was performed. In these conditions, IT1t was shown to reduce type I IFN in cells transfected with the control siRNA (siCTR) but lost its biological activity in CXCR4 siRNA-treated cells stimulated with HIV X4 or HIV R5. Thus, IT1t inhibited type I IFN through CXCR4 engagement, similarly to endogenous amines. Then it has been evaluated whether the well know CXCR4 antagonist AMD3100 could inhibit interferon production on HIV-stimulated pDC. Interestingly, it confirmed that AMD3100 alone did not block type I IFN nor TRAIL expression by HIV-activated pDC, suggesting a different mechanism of action than IT1t and other amines. Then it has been tested whether AMD3100 is able to block amine action by limiting the access of IT1t pocket. Indeed, AMD-3100 binding site overlapped the identified amine binding pocket. The expression of TRAIL, IFN-α and IFN-β were also quantified in pDC treated or not with AMD3100. Purified cells were pre-incubated with AMD3100 for 1 hour and then followed by histamine or CB for 1 hour and finally exposed to HIV-1 overnight. AMD3100 drastically abolished biological activities of histamine and CB on HIV activated-pDC. Indeed, AMD3100 treatment could restore type I IFN mRNA and protein, and TRAIL productions inhibited by histamine or CB in HIV-activated pDC. These results were confirmed on a panel of pDC cytokine secretion (IFN-γ and IL6) and ISG (ISG56). Altogether, these results unambiguously demonstrate that CXCR4 is required for the inhibitory activity of amines on pDC activation.

9. Mab 12G5 Inhibits HIV-Induced Production of Type I Interferons

The inventors could show that the 12G5 monoclonal antibody inhibits IFN-I production (FIG. 8).

10. IFN-II Secretion by NK Cells is Inhibited by Amines

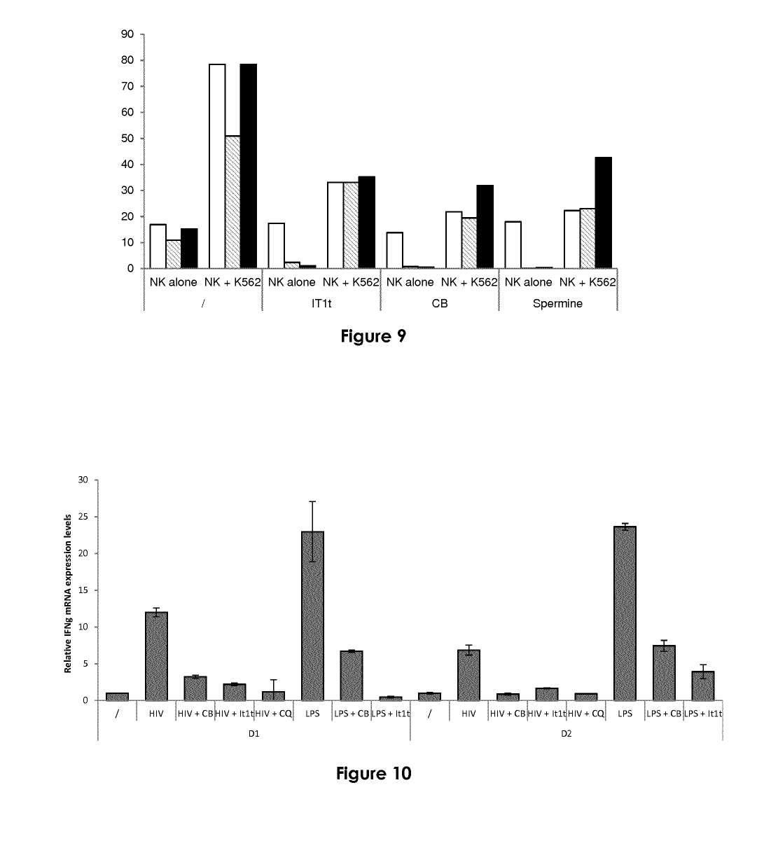

The expression level of IFN-γ, TNF-α and CD107aby NK cells in the absence or presence of It1t, clobenpropit (CB) and spermine was measured by flow cytometry (FIG. 9).

The inventors could thus show that It1T, clobenpropit (CB) and spermin decrease IFN-γ, TNF-α and CD107a expression by NK cells activated by K562 cells.

11. IFN-II Secretion by Monocytes is Inhibited by Amines

Monocytes were pre-incubated with clobenpropit (CB), IT1t or chloroquine before being activated by HIV and LPS. IFN-γ levels were measured by RT-qPCR and normalized to RPL13A mRNA expression.

FIG. 10 thus shows that clobenpropit (CB), IT1t and chloroquine inhibit IFN-γ expression by HIV or LPS stimulated monocytes.

### EXAMPLE II: ANTI-ARTHRITIC EFFICACY OF TEST COMPOUNDS IN A THERAPEUTIC COLLAGEN-INDUCED ARTHRITIS MODEL IN DBA1/J MICE

A. Materials and Methods

1. Animals

80 DBA1/J mice (male, 7-8 weeks) was received and placed in quarantine for 3 days with daily inspections. Ear tag mice for individual identification.

2. Protocol

Prepare 0.01M acetic acid by addition of 0.1 ml glacial acetic acid to 160 ml deionized water.

Prepare bovine Type II collagen solution by dissolving at 4 mg/ml in 0.01M acetic acid at 4-8° C. with stirring overnight.

Day −1:

Prepare immunogen by emulsifying a 1:1 vol:vol combination of collagen solution and Complete Freund's Adjuvant (CFA) (M. tuberculosis H37Ra suspension: 4 mg/ml).

Day 0:

Individual mouse weights were recorded. Hind paw thickness were recorded by digital caliper. 80 mice subcutaneous were injected with collagen/CFA emulsion (0.05 ml/mouse; 100 μg/mouse collagens in CFA) using a 1 ml syringe fitted with a 25G needle. Mice returned to cages.

Day 20:

Prepare bovine collagen Type II by dissolving at 4 mg/ml in 0.01 M acetic acid at 4-8° C. with stirring overnight.

Day 21:

80 mice were boosted with collagen/ICFA emulsion. Then their individual weights were recorded.

Prepare immunogen by emulsifying (homogenizer) a 1:1 vol:vol combination of collagen solution and Incomplete Freund's Adjuvant (ICFA).

Inject subcutaneous immunogen (0.050 ml/mouse; 100 μg/mouse collagen in ICFA) using a 1 ml syringe fitted with a 25G needle. Then mice returned to cages.

Day 28:

Selection of mice for assignment to groups for therapeutic dosing.

This selection took place a few days earlier or later than Day 28 depending on how arthritis had developed in the animals.

Mice were scored for signs of arthritis:

1) Each paw receives a score

2) 0=no visible effects of arthritis

3) 1=edema and/or erythema of 1 digit

4) 2=edema and/or erythema of 2 digits

5) 3=edema and/or erythema of more than 2 digits

6) 4=severe arthritis of entire paw and digits

7) Calculate Arthritic Index (AI) by addition of individual paw scores and record.

8) Maximum AI=16

Mice with an AI score within a range of 2-6 were selected for assignment to groups for therapeutic dosing as in Table 4.

Selected mice (from a total of 100 immunized with collagen) were assigned to 8 groups (N=8) so that each group has approximately the same Group Mean AI.

Begin intraperitoneal (IP) dosing once daily (QD) according to Table 4.

The compounds clobenpropit (CB) and IT1t were tested. CB and IT1t were stored at 4° C. The compounds were prepared freshly before the treatment by solubilization in PBS (solubility is >50 mg/ml in water for both compounds). These compounds were doses 7 days per week (Saturday and Sunday included) daily for 14 days.

TABLE 4
Therapeutic group treatment

| Group | No. Mice | Treatment | Dose (mg/kg)* |
|---|---|---|---|
| 1 | 8 | PBS | 10 |
| 2 | 8 | Prednisolone | 3 |
| 3 | 8 | IT1† | 3 |
| 4 | 8 | IT1† | 10 |
| 5 | 8 | IT1† | 30 |
| 6 | 8 | Clobenpropit | 3 |
| 7 | 8 | Clobenpropit | 10 |
| 8 | 8 | Clobenpropit | 30 |

*Single injection on Day 28 to 42.

Days 28-42:

Mice were weighed, scored for signs or arthritis, and hind paw thickness is measured by digital caliper three-times weekly (Monday, Wednesday and Friday). Any adverse reactions to treatment were recorded.

Termination: AI score were recorded for each limb. The paw thickness of the hind limbs is measured with digital caliper. Mice were anesthetized and exsanguinated into pre-chilled EDTA-tubes.

1) Blood was processed to plasma which was stored at −80° C. in four labeled Eppendorf tubes.

2) Plasma was assayed by ELISA for IL-1β, IL-6, TRAIL. Then, all limbs were collected.

1) After removal, the limbs were fixed individually in 10% neutral buffered formalin for possible histopathology.

B. Results

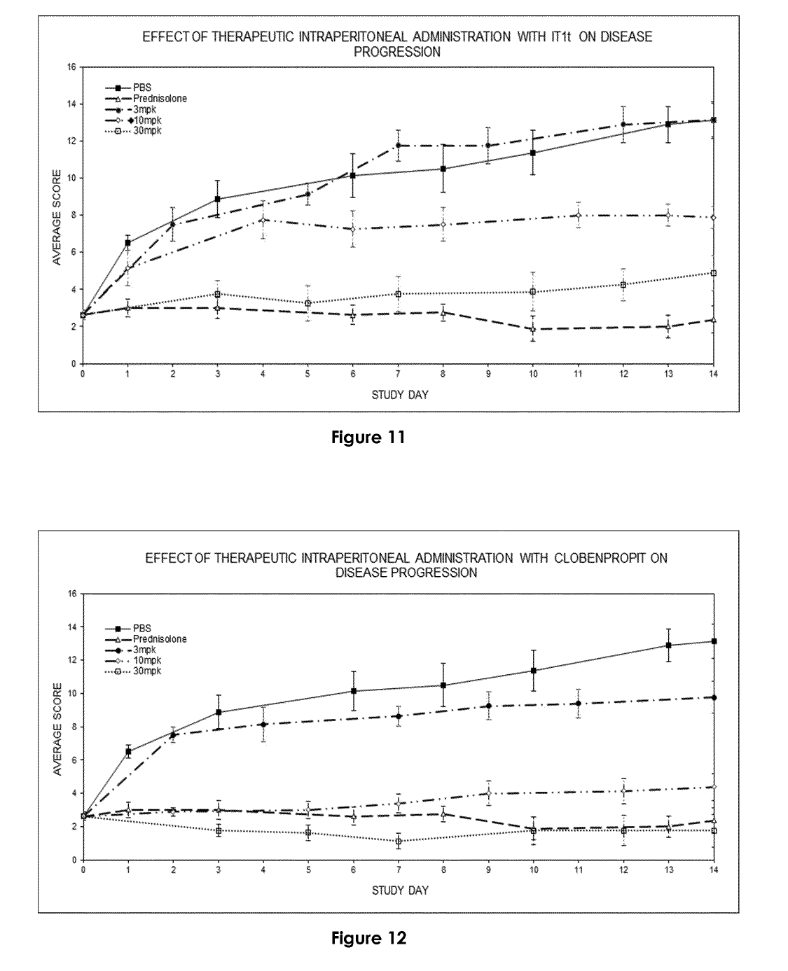

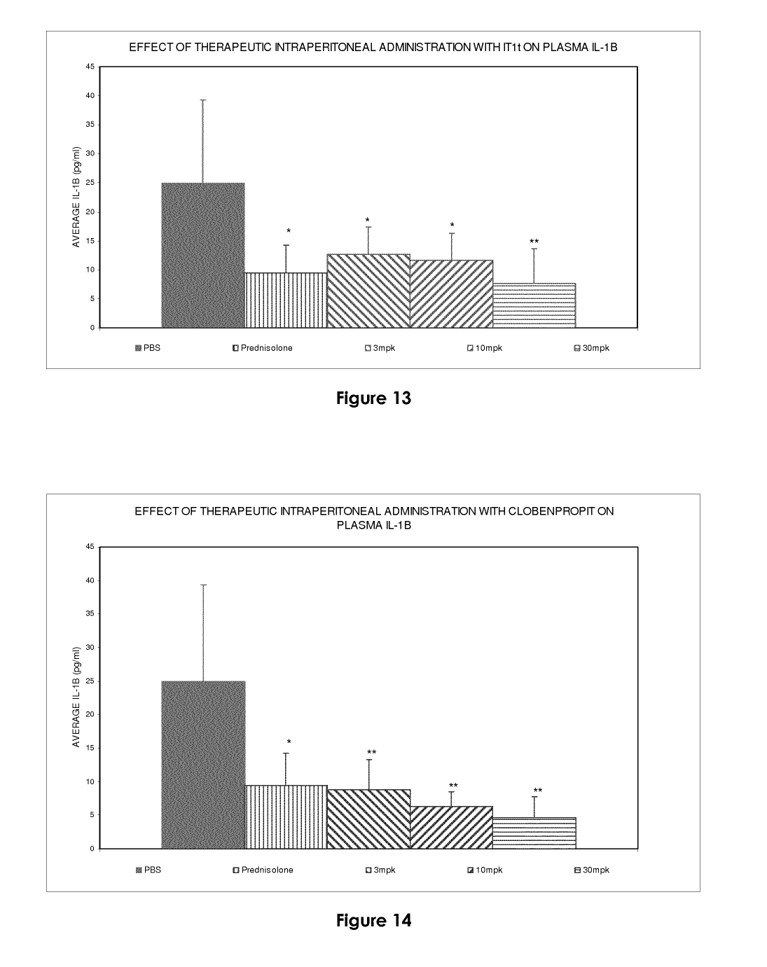

Daily therapeutic treatment by intraperitoneal injection of IT1t (FIG. 11 and FIG. 13) resulted in a significant dose dependent reduction in the signs of disease, as well as plasma concentrations of IL-1β and IL-6. TRAIL was significantly inhibited regardless of dose.

Therapeutic treatment by daily intraperitoneal injection of clobenpropit (FIG. 12 and FIG. 14) resulted in a significant dose-dependent reduction in the signs of disease, as well as plasma concentrations of IL-1β, IL-6 and TRAIL (FIG. 18).

1. Disease Development

As animals developed disease, they were sorted into treatment groups of eight mice each with AI in the range of 2-4 and an average group AI of 2.6, prior to initiation of the dosing regimen. Disease appeared to develop first in the hind limbs, probably due to the fact that the animals spent more time standing on their hind limbs, alone, than they do on all four limbs. Once daily intraperitoneal injection with PBS (Group 1) yielded an AI of 13.1 on Day 42 (fourteen days of dosing. At the termination of the study, the diseased mice had plasma levels of 25 pg/ml of IL-1β, 156 pg/ml of IL-6, and 328 pg/ml of TRAIL.

2. Therapeutic Treatment with Prednisolone (Group 2)

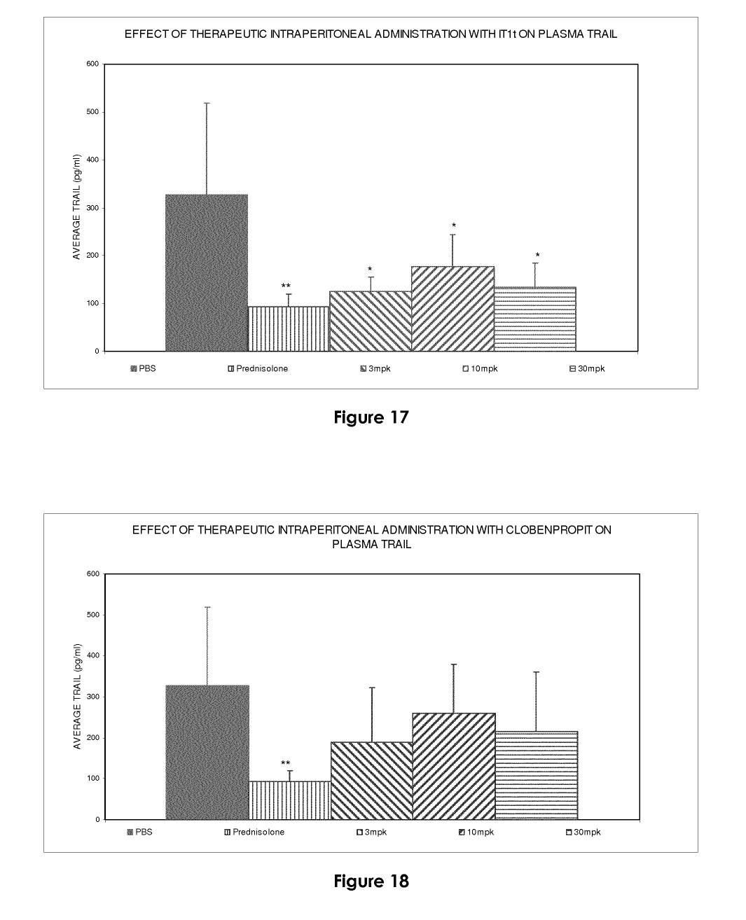

Daily intraperitoneal injection with 3 mg/kg prednisolone starting on Day 28 resulted in an immediate arrest in disease progression yielding an 82% inhibition of disease severity on Day 42 (average AI=2.4). As positive control of this study, this treatment regimen significantly reduced plasma levels of IL-1β (62%), IL-6 (79%), and TRAIL (72%) (FIG. 17).

3. Therapeutic Treatment with IT1t (Groups 3-5)

Daily intraperitoneal injection with IT1t resulted in a dose-dependent inhibition of the disease progression. At the lowest dose (3 mg/kg, Group 3) no amelioratory effect was observed in the macroscopic disease score (average AI=13.1) at the termination of the study. However, this treatment regimen did yield a significant reduction in terminal plasma concentrations of IL-1β (49%) (FIG. 13) and TRAIL (62%) (FIG. 17).

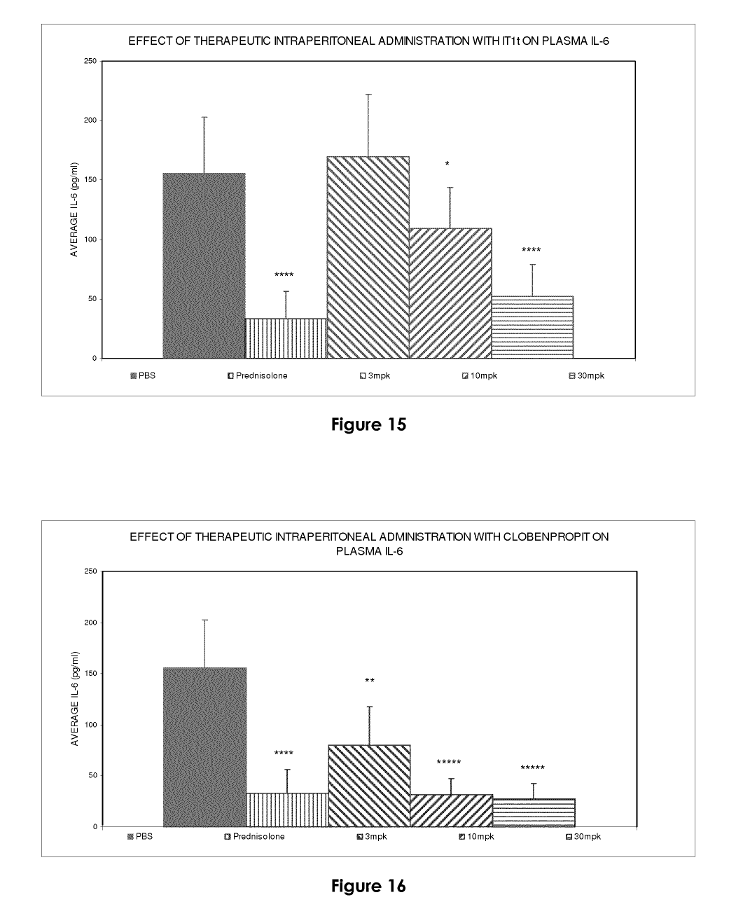

At the intermediate dose (10 mg/kg, Group 4), the rate of disease progression was attenuated, yielding a significant 40% reduction in disease severity (average AI=7.9) at the termination of the study. This treatment regimen also yielded a significant reduction in terminal plasma concentrations of IL-1β (53%) (FIG. 13), IL-6 (30%) (FIG. 15), and TRAIL (46%) (FIG. 18).

At the highest dose (30 mg/kg, Group 5) the rate of disease progression was severely curtailed, yielding a significant 63% reduction in the terminal disease severity (AI=4.9). This treatment regimen also yielded a significant reduction in terminal plasma concentrations of IL-1β (69%) (FIG. 13), IL-6 (66%) (FIG. 15), and TRAIL (51%) (FIG. 18).

4. Therapeutic Treatment with Clobenpropit (Groups 6-8)

Daily intraperitoneal injection with clobenpropit resulted in a dose-dependent inhibition of the disease progression. At the lowest dose (3 mg/kg, Group 6) a gradual reduction in the rate of disease progression was observed, yielding a significant 25% inhibition of the disease severity (average AI=9.8) at the termination of the study. This treatment regimen also yielded a significant reduction in terminal plasma concentrations of IL-1β (64%) (FIG. 14) and IL-6 (49%) (FIG. 16). This treatment regimen had also an effect on terminal plasma concentration of TRAIL (FIG. 18).

At the intermediate dose (10 mg/kg, Group 7), the rate of disease progression was severely curtailed, yielding a significant 66% reduction in disease severity (average AI=4.4) at the termination of the study. This treatment regimen also yielded a significant reduction in terminal plasma concentrations of IL-1β (75%) (FIG. 14) and IL-6 (80%) (FIG. 16). This treatment regimen had also an effect on terminal plasma concentration of TRAIL (FIG. 18).

At the highest dose (30 mg/kg, Group 8) initiation of treatment resulted in an immediate reversal of the signs of disease such that at the termination of the study a significant 87% reduction in disease severity (average AI=1.8) was recorded. This treatment regimen also yielded a significant reduction in terminal plasma concentrations of IL-1β (82%) (FIG. 14), (82%) (FIG. 16) and TRAIL (FIG. 18).

### EXAMPLE III: ANTI-INFLAMMATORY EFFICACY OF THE COMPOUNDS CLOBENPROPIT AND LILT IN A PRISTANE-INDUCED SYSTEMIC LUPUS ERYTHEMATOSUS (SLE) MODEL

A. Materials and Methods

1. Animals

Receive and quarantine (SOP 560) 70 female Balb/C (20-25 g) mice and house in filter-topped cages supplied with autoclaved bedding.

Examine mice once daily during 72-hour quarantine period and record any signs of clinical distress, disease or injury. Animals exhibiting no signs are accepted for the Study.

Accepted animals are transferred to routine maintenance and housed at 8 per cage. The treatment groups are identified by cage card.

The animals are weighed, ear tagged for individual identification and randomly assigned to 8 treatment groups of 8 animals each and two groups of 3 animals (for pre-tolerance at 30 mg/kg of survival).

Induction of SLE by intraperitoneal injections of pristane and treatment regimens with compounds are shown in Table 5.

2. Test Compounds

The test items compounds clobenpropit (CB) and IT1t are stored at 4° C. The compounds are prepared freshly before the treatment by solubilization in PBS (vehicle) (solubility is >50 mg/ml in PBS for both compounds).

TABLE 5
Group treatment

| Group | Mice | Material | Dose | | ROA | Frequency |
|---|---|---|---|---|---|---|
| 1 | 8 | Pristane + | 0.5 | ml | IP* | Single dose** |
| | | PBS | 10 | (mg/kg) | IP | QD*** |
| 2 | 8 | Pristane + | 0.5 | ml | IP | Single dose |
| | | Prednisolone | 15 | (mg/kg) | PO**** | QD |
| 3 | 8 | Pristane + | 0.5 | ml | IP | Single dose |
| | | CB | 3 | (mg/kg) | IP | QD |
| 4 | 8 | Pristane + | 0.5 | ml | IP | Single dose |
| | | CB | 10 | (mg/kg) | IP | QD |
| 5 | 8 | Pristane + | 0.5 | ml | IP | Single dose |
| | | CB | 30 | (mg/kg) | IP | QD |
| 6 | 8 | Pristane + | 0.5 | ml | IP | Single dose |
| | | IT1† | 3 | (mg/kg) | IP | QD |
| 7 | 8 | Pristane + | 0.5 | ml | IP | Single dose |
| | | IT1† | 10 | (mg/kg) | IP | QD |
| 8 | 8 | Pristane + | 0.5 | ml | IP | Single dose |
| | | IT1† | 30 | (mg/kg) | IP | QD |

ROA = Route of administration
*IP = intraperitoneal injection
**Single injection on Day 0
***Test compounds are dosed 7 days per week (Saturday and Sunday included), daily; for 10 weeks. On day 0, the dose of test compound is given 1 hour following Pristane injection
****PO = oral dosing

3. Monitoring/Measuring Parameters

Mouse weights are recorded twice a week, and daily observations for clinical signs.

Sera collections:

Groups 1-8: Predose and weeks 4

Collected sera are stored at −80° C. for measurements of autoantibody on weeks 4, 8 and 10. (Predose will kept at −80° C. for potential use). Anti-dsDNA level is a standard screening readout for identifying efficacy of test compounds in a SLE model.

Measurements: weeks 4, 8, 10: (1) autoantibody (anti-dsDNA), collagen and cytokines TNFα, IL-6 and TRAIL by ELISA and (2) ANA antibody by IFA.

Termination (week 10): all mice are anesthetized (SOP1810) and exsanguinated (SOP 1687). Blood is collected and processed for serum (SOP 6001) and stored at −80° C. for measurements of autoantibody, collagen and cytokines.

Spleen and Kidneys take down: Spleens and kidneys are taken down and fixed in 10% neutral buffered formalin for potential use.

4. Results

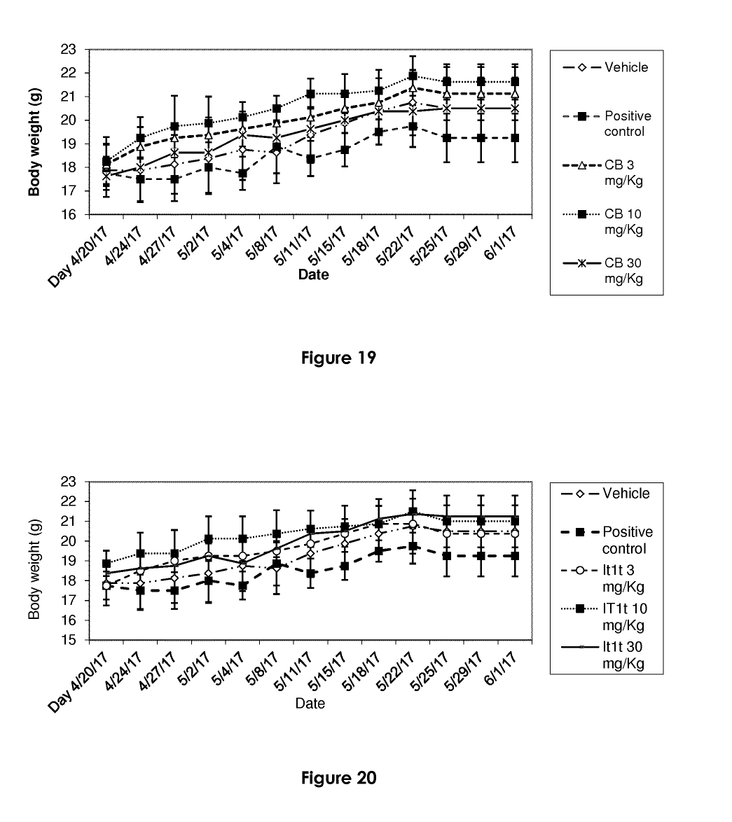

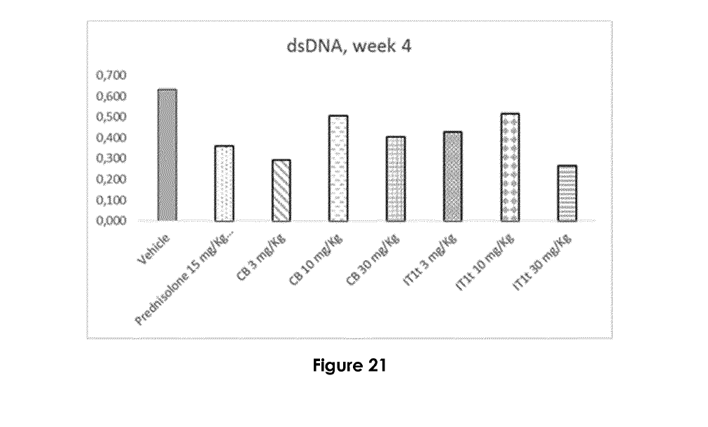

Mice treated with CB (FIG. 19) and IT1t (FIG. 20) show no loss of body weight during the study compared to dose started date. The results indicate test compounds have no toxicity in terms body weight loss; and have the potential to be used for chronic treatments. Besides, observations indicate that CB and IT1 t reduce symptoms of pristane-Induced Systemic Lupus Erythematosus in treated mice. Indeed, there is inhibition of ds-DNA level in test compounds (CB, IT1) compared to group 1 (vehicle) (FIG. 21).

1. A CXCR4 receptor-binding compound for use for decreasing interferon (IFN) level in an individual, provided the CXCR4 receptor-binding compound is different from histamine.

2. The CXCR4 receptor-binding compound for use according to claim 1, wherein the compound comprises from 1 to 45 carbon atoms and at least one amine group positively charged at a pH from 6 to 8.

3. A CXCR4 receptor-binding compound for use according to claim 1, wherein the CXCR4 receptor-binding compound interacts with at least 8 amino acids of a CXCR4 receptor represented by SEQ ID NO: 1, wherein the amino acids are selected from the group consisting of tryptophan 94, tryptophan 102, aspartic acid 97, aspartic acid 187, tyrosine 116, tyrosine 190, arginine 183, isoleucine 185, valine 112, cysteine 186 and glutamic acid 288.

4. The CXCR4 receptor-binding compound for use according to claim 1, wherein the compound is selected from the group consisting of:
serotonin, dopamine, L-dopamine, spermine, or spermidine,
an anti-CXCR4 receptor antibody, antibody fragment, scFv antibody, or aptamer,
a compound of the following formula (I): 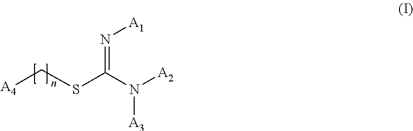
wherein:
n is an integer from 1 to 6,
A₁, A₂ and A₃, which may be identical or different, represent:
a hydrogen atom, or
an alkyl group having from 1 to 12 carbon atoms, optionally substituted by at least one hydroxyl group, a halogen atom, a carbonitril group, a trifluoromethyl group, an amine group, a urea, or an O-alkyl or S-alkyl group having from 1 to 12 carbon atoms, or
a heterocycle, heteroaryl, aryl, arylalkyl or alkylaryl group having from 3 to 12 carbon atoms, optionally substituted by at least one hydroxyl group, a halogen atom, a carbonitril group, a trifluoromethyl group, an amine group, a urea, or an O-alkyl or S-alkyl having from 1 to 12 carbon atoms; and
A₄ represents an aryl, arylalkyl or alkylaryl group having from 3 to 20 carbon atoms optionally substituted by at least one hydroxyl group, a halogen atom, a carbonitril group, a trifluoromethyl group, an amine group, a urea group, or an O-alkyl or S-alkyl group having from 1 to 12 carbon atoms;
or a pharmaceutically acceptable salt and/or hydrate thereof;
a compound of the following formula (II): 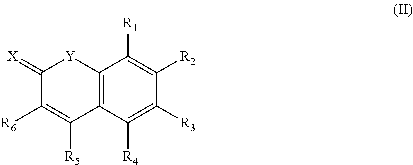
wherein
R₁, R₂, R₃, and R₄, which may be identical or different, represent a hydrogen atom, a halogen atom, a hydroxyl group, an alkyl group having from 1 to 12 carbon atoms, optionally substituted by at least one hydroxyl group, an amine group or a halogen atom, wherein R₁ and R₂, and/or R₂ and R₃ and/or R₃ and R₄ can be included in a same cycle;
X and Y, which may be identical or different, represent S or O;
R₅ and R₆, which may be identical or different, represent a hydrogen atom or an alkyl group having from 1 to 5 carbon atoms substituted by at least one amine group, provided at least one of R₅ and R₆ represents an alkyl group having from 1 to 5 carbon atoms substituted by at least one amine group;
or a pharmaceutically acceptable salt and/or hydrate thereof,
a compound of the following formula (III): 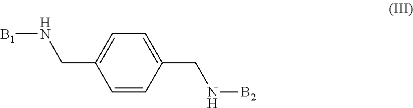
wherein
B₁ and B₂ which are identical or different, represent:
an aryl or heteroaryl group having from 3 to 6 carbon atoms, optionally substituted by a hydroxyl group, a halogen atom, an alkoxy group, a thioalkoxy group, a CF₃ group, a CN group, a —NR₇R₈ group, an amide or an alkyl, S-alkyl or O-alkyl group having from 1 to 6 carbon atoms, or
a cycloalkyl or heterocycloalkyl group having from 3 to 6 carbon atoms, optionally substituted by, a hydroxyl group, a halogen atom, an alkoxy group, a thioalkoxy group, a CF₃ group, a CN group, a —NR₇R₈ group or an alkyl, S-alkyl or O-alkyl group having from 1 to 6 carbon atoms,
wherein R₇ and R₈ which are identical or different, represent a hydrogen atom, an alkyl group having from 1 to 6 carbon atoms or a heterocycloalkyl group having from 3 to 6 carbon atoms;
or a pharmaceutically acceptable salt and/or hydrate thereof;
a compound of the following formula (IV): 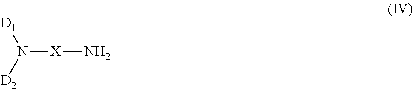
wherein
D₁ and D₂, which may be identical or different, represent:
an alkyl group having from 1 to 6 carbon atoms, optionally substituted by at least one hydroxyl group, a halogen atom, a CF₃ group, a CN group, an amine group, or an alkyl, O-alkyl or S-alkyl group having from 1 to 12 carbon atoms, or
an aryl, heteroaryl, cycloalkyl, or heterocycloalkyl group having from 3 to 12 carbon atoms, optionally substituted by at least one hydroxyl group, a halogen atom, a CF₃ group, a CN group, an amine group, or an alkyl, O-alkyl or S-alkyl group having from 1 to 12 carbon atoms; or
D₁ and D₂ are linked together to form a N-containing aryl or heteroaryl group having from 3 to 12 carbon atoms and optionally substituted by at least one amine group optionally substituted by an alkylheteroaryl group having from 3 to 12 carbon atoms, and
X represents:
an alkyl group having from 1 to 6 carbon atoms, or
—R₉—Y—R₁₀— wherein, R₉ and R₁₀ which are identical or different represent an alkyl group having from 1 to 6 carbon atoms and Y represents an aryl or heteroaryl group having from 3 to 6 carbon atoms, optionally substituted by a halogen atom, a hydroxyl group, an amide group, an amine group, an alkoxy group, an ester group, a CF₃ group, a CN group or an alkyl, O-alkyl or S-alkyl group having from 1 to 6 carbon atoms optionally substituted by a hydroxyl group, an amine group or an O-alkyl group having from 1 to 6 carbon atoms;
or a pharmaceutically acceptable salt thereof and/or hydrate thereof.

5. The CXCR4 receptor-binding compound for use according to claim 1, wherein the CXCR4 receptor-binding compound is selected from the group consisting of IT1t, clobenpropit, AMD070, FFN102, FFN202, and FFN511.

6. The CXCR4 receptor-binding compound for use according to claim 1, wherein the interferon (IFN) is selected from the group consisting of a type I interferon (IFN-I), a type II interferon (IFN-II) and a type III interferon (IFN-III).

7. The CXCR4 receptor-binding compound for use according to claim 1, for inhibiting IFN secretion by immune cells.

8. The CXCR4 receptor-binding compound for use according to claim 1, for inhibiting IFN secretion by immune cells selected from the group consisting of plasmacytoid dendritic cells, monocytes and Natural Killer (NK) cells.

9. The CXCR4 receptor-binding compound for use according to claim 1, in the prevention or treatment of interferonopathies or autoimmune diseases.

10. The CXCR4 receptor-binding compounds for use according to claim 1, in the prevention or treatment of a disease selected from the group consisting of Aicardi-Goutières syndrome, familial chilblain lupus, spondyenchondromatosis, psoriasis, Systemic lupus erythematosus, Sting-associated vasculopathy, crohn disease, Proteasome-associated auto-inflammatory syndrome (PRAAS), Singleton-Merten syndrome, Sjogren's syndrome, myositis, systemic sclerosis, type I diabetes mellitus, autoimmune thyroid disease, rheumatoid arthritis, multiple sclerosis, and atherosclerosis.

11. The CXCR4 receptor-binding compound for use according to claim 1, wherein the individual has a chronic viral infection.

12. The in vitro use of a CXCR4 receptor-binding compound as defined in any claim 1, for inhibiting IFN secretion by immune cells, provided the CXCR4 receptor-binding compound is different from histamine.

13. An in vitro screening method for identifying compounds for decreasing IFN level in an individual from candidate compounds, wherein the candidate compounds are CXCR4 receptor-binding compounds as defined in claim 1.

14. The in vitro screening method according to claim 13, comprising the steps of:
contacting blood cells with a candidate compound;
determining the level of secretion of IFN by the contacted blood cells;
selecting the candidate compound which decreases the level of secretion of IFN with respect to the level of secretion of IFN before the blood cells have been contacted by the candidate compound, thereby identifying a compound for decreasing IFN level.

15. An in vitro screening method for identifying compounds for decreasing IFN level in an individual from candidate compounds, comprising:
binding a CXRC4 receptor with a detectable CXCR4 receptor-binding compound as defined in claim 1;
contacting the CXCR4 receptor bound to the detectable CXCR4 receptor-binding compound with a candidate compound;
selecting the candidate compound which decreases the binding of the detectable CXCR4 receptor-binding compound to the CXCR4 receptor, thereby identifying a compound for decreasing IFN level.

16. An in silico method for screening compounds useful for decreasing IFN level in an individual from candidate compounds, or for designing compounds useful for decreasing IFN level in an individual, comprising a computer-implemented step of determining if a designed compound or a candidate compound interacts with at least 8 amino acids of a CXCR4 receptor represented by SEQ ID NO: 1, wherein the amino acids are selected from the group consisting of tryptophan 94, tryptophan 102, aspartic acid 97, aspartic acid 187, tyrosine 116, tyrosine 190, arginine 183, isoleucine 185, valine 112, cysteine 186 and glutamic acid 288.


SEQUENCE LISTING (35 sequences)

SEQ ID NO: 1 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 2 - DNA, 23 bp, Artificial
  PCR primer
cctggaggagaagaggaaagaga

SEQ ID NO: 3 - DNA, 25 bp, Artificial
  PCR primer
ttgaggacctctgtgtatttgtcaa

SEQ ID NO: 4 - DNA, 21 bp, Artificial
  PCR primer
gctgaagcagatgcaggacaa

SEQ ID NO: 5 - DNA, 21 bp, Artificial
  PCR primer
tgacggagttgccacttgact

SEQ ID NO: 6 - DNA, 20 bp, Artificial
  PCR primer
ccagttccagaaggctccag

SEQ ID NO: 7 - DNA, 20 bp, Artificial
  PCR primer
tcctcctgcatcacacaggc

SEQ ID NO: 8 - DNA, 21 bp, Artificial
  PCR primer
cccacagcctgggtaatagga

SEQ ID NO: 9 - DNA, 21 bp, Artificial
  PCR primer
cagcagatgagtcctctgtgc

SEQ ID NO: 10 - DNA, 21 bp, Artificial
  PCR primer
tgcattacctgaaggccaagg

SEQ ID NO: 11 - DNA, 21 bp, Artificial
  PCR primer
agcaattgtccagtcccagtg

SEQ ID NO: 12 - DNA, 20 bp, Artificial
  PCR primer
ggacgccttggaagagtcac

SEQ ID NO: 13 - DNA, 20 bp, Artificial
  PCR primer
ctggtctaggacgtcctcca

SEQ ID NO: 14 - DNA, 20 bp, Artificial
  PCR primer
gggcctgtatccagcctcag

SEQ ID NO: 15 - DNA, 20 bp, Artificial
  PCR primer
gaggaggcggaagaggttga

SEQ ID NO: 16 - DNA, 21 bp, Artificial
  PCR primer
ggcagccaacctaagcaagat

SEQ ID NO: 17 - DNA, 21 bp, Artificial
  PCR primer
cagggtcacctgacacattca

SEQ ID NO: 18 - DNA, 20 bp, Artificial
  PCR primer
taaccacccctgacccaacc

SEQ ID NO: 19 - DNA, 20 bp, Artificial
  PCR primer
atttgccgaagagccctcag

SEQ ID NO: 20 - DNA, 22 bp, Artificial
  PCR primer
aagggccaagagaatatccgaa

SEQ ID NO: 21 - DNA, 22 bp, Artificial
  PCR primer
actagggttgccagatttaaca

SEQ ID NO: 22 - DNA, 22 bp, Artificial
  PCR primer
aagggccaagagaatatccgaa

SEQ ID NO: 23 - DNA, 20 bp, Artificial
  PCR primer
gctggccacagctttcaaga

SEQ ID NO: 24 - DNA, 20 bp, Artificial
  PCR primer
aagaagagctggctatggca

SEQ ID NO: 25 - DNA, 20 bp, Artificial
  PCR primer
tcatgttccatgctgctgac

SEQ ID NO: 26 - DNA, 20 bp, Artificial
  PCR primer
aggacaggaagctgaaggag

SEQ ID NO: 27 - DNA, 20 bp, Artificial
  PCR primer
agtgggtgtttcctgcaagg

SEQ ID NO: 28 - DNA, 20 bp, Artificial
  PCR primer
cgctgtacctgcatcagcat

SEQ ID NO: 29 - DNA, 22 bp, Artificial
  PCR primer
gcaatgatctcaacacgtggac

SEQ ID NO: 30 - DNA, 19 bp, Artificial
  PCR primer
cagcgggatgactttccaa

SEQ ID NO: 31 - DNA, 20 bp, Artificial
  PCR primer
aggcaagatttggacctgca

SEQ ID NO: 32 - DNA, 22 bp, Artificial
  PCR primer
gcatgacggacaagtacaggct

SEQ ID NO: 33 - DNA, 21 bp, Artificial
  PCR primer
aaagtaccagtttgccacggc

SEQ ID NO: 34 - DNA, 20 bp, Artificial
  PCR primer
acacgctgttcgaatgggat

SEQ ID NO: 35 - DNA, 23 bp, Artificial
  PCR primer
tcgatcatagctgatgaggacaa